(19) United States
(12) Patent Application Publication
(10) Pub. No.: US 2023/0212510 A1
(43) Pub. Date: Jul. 6, 2023

(54) METHODS FOR ORGANOIDS PRODUCTION

(71) Applicant: CITY OF HOPE, Duarte, CA (US)

(72) Inventors: Mark LABARGE, Duarte, CA (US); Michael TODHUNTER, Duarte, CA (US)

(73) Assignee: CITY OF HOPE, Duarte, CA (US)

(21) Appl. No.: 17/998,011

(22) PCT Filed: Apr. 22, 2021
(86) PCT No.: PCT/US21/28619

§ 371 Date: Nov. 4, 2022

Related U.S. Application Data
(60) Provisional application No. 63/019,793, filed on May 4, 2020.

(51) Int. Cl. C12N 5/071

(52) U.S. Cl. CPC C12N 5/0631; C12N 2513/00; C12N 2531/00; C12N 2533/76

(57) ABSTRACT

Disclosed are methods of producing organoids in the absence of any exogenous extracellular matrix or in the presence of an exogenous extracellular matrix at a concentration lower than gelling concentration, using microcontainers sealed with a hydrogel lid as well as organoids produced by this technique.

### PRIORITY CLAIM

This application claims priority to U.S. Provisional Patent Application No. 63/019,793, filed on May 4, 2020, the content of which is hereby incorporated by reference in its entirety.

### STATEMENT REGARDING FEDERALLY SPONSORED RESEARCH OR DEVELOPMENT

This invention was made with government support under Grant Number BC141351, awarded by the Department of Defense Army Medical Research Acquisition Activity and Grant Number P30CA033572, awarded by the National Institutes of Health. The government has certain rights in the invention.

### BACKGROUND

Organoid culture is a leading approach to obtain physiological data from human cells in the laboratory setting. Organoids provide their constituent cells with the microenvironment cues necessary to elicit native structure and function. Consequently, organoids can be better physiological models of biological tissue than cells grown on plastic, which has made organoid culture the subject of much research and development (Sachs et al., 2018). Nevertheless, organoid culture faces limitations to widespread and useful deployment. One of the most important limitations of organoid culture is its need for exogenous extracellular matrix scaffolds.

Exogenous scaffolds, ranging from semi-synthetic polymer-peptide conjugates (Cruz-Acuña and Garcia, 2017) to jellied secretions from cancer cells (Lutolf and Gjorevski, 2018), are typically required for cells in three-dimensional culture to survive and develop into organoids. For mammary organoids, the typical scaffold is laminin-rich extracellular matrix (IrECM), such as Matrigel, which contains components of the epithelial basement membrane, as well as stromal components (Hansen et al., 2009). Although IrECM permits mammary organoids to recapitulate various physiological behaviors (Cerchiari et al., 2015; Todhunter et al., 2015), it has well-known limitations, including lot-to-lot variability, high cost, and discrepancies in both composition (Hansen et al., 2009) and structure (Kleinman et al., 1986) from bona fide basement membrane. These problems have driven a market for IrECM substitutes, but no substitute has yet been devised that emulates all aspects of physiological matrix. Scaffold-free cultures can bypass these issues for some cell types but are infeasible for anchorage-dependent cells, including human mammary epithelial cells (HMECs), which undergo anoikis under scaffold-free conditions (Hindupur et al., 2014).

Accordingly, there is a need in the field to develop organoids without using any exogenous extracellular matrix or at a low concentration of exogenous extracellular matrix. This disclosure provides a novel organoid culture method that satisfies the need.

### SUMMARY OF THE INVENTION

In one aspect, disclosed herein is a method of producing an organoid without any exogenous extracellular matrix. The method comprises the steps of loading organ-specific cells in a microcontainer containing a culturing medium, overlaying a hydrogel over the culture containing the cells such that the hydrogel forms a lid which is in direct contact with the surface of the culture to seal the culture; and culturing the cells in the hydrogel-sealed microcontainer to obtain the organoid, wherein the culturing medium does not contain any exogenous extracellular matrix. In certain embodiments, the cells include epithelial cells and fibroblast cells. In certain embodiments, the culturing medium has a higher density than the hydrogel lid. In certain embodiments, the culturing medium has a density between about 1.1 g/ml and about 1.2 g/ml and the hydrogel lid has a density of about 1.0 g/ml. In certain embodiments, the culturing medium comprises one or more biological colloids to achieve a higher density than the lid. In certain embodiments, the biological colloids include dextrin, maltodextrin, albumin, PEG-8000 and hydroxyethyl starch. In certain embodiments, the albumin includes bovine serum albumin or bovine serum albumin, fraction V. In certain embodiments, the hydrogel comprises agarose.

In another aspect, disclosed herein is a method of producing an organoid with a low concentration of exogenous extracellular matrix. The method comprises the steps of loading organ-specific cells in a microcontainer containing a culturing medium, overlaying a hydrogel over the culture containing the cells such that the hydrogel forms a lid which is in direct contact with the surface of the culture to seal the culture; and culturing the cells in the hydrogel-sealed microcontainer to obtain the organoid, wherein the culturing medium contains a low concentration of exogenous extracellular matrix, wherein the low concentration of exogenous extracellular matrix is lower than its minimum gelling concentration and insufficient to form a gel in the culturing medium. In certain embodiments, the culturing medium contains 0.5 mg/mL-1 mg/mL Matrigel. In certain embodiments, the cells include epithelial cells and fibroblast cells. In certain embodiments, the culturing medium has a higher density than the hydrogel lid. In certain embodiments, the culturing medium has a density between about 1.1 g/ml and about 1.2 g/ml and the hydrogel lid has a density of about 1.0 g/ml. In certain embodiments, the culturing medium comprises one or more biological colloids to achieve a higher density than the lid. In certain embodiments, the biological colloids include dextrin, maltodextrin, albumin, PEG-8000 and hydroxyethyl starch. In certain embodiments, the albumin includes bovine serum albumin or bovine serum albumin, fraction V. In certain embodiments, the hydrogel comprises agarose.

In another aspect, this disclosure relates to an organoid produced by the methods disclosed above. In certain embodiments, the organoid exhibits contractility. In certain embodiments, the organoid exhibits pulsatile contractility.

In yet another aspect, this disclosure relates to a microcontainer for organoid culturing in the absence of any exogenous extracellular matrix, comprising walls composed of a hydrogel material and a hydrogel lid, wherein once cells and culturing medium are loaded in the microcontainer, the hydrogel walls and lid prevent the cells from escaping but allow air and liquid exchange with the environment. In certain embodiments, the hydrogel material includes agarose, gellan, alginate hydrogels or a combination thereof. In certain embodiments, the microcontainer has a diameter between about 100 μm and 150 μm. In certain embodiments, the microcontainer has a depth between about 100 μm and 350 μm. In certain embodiments, the microcontainer has a diameter of about 100 μm and a depth of about 200 μm. In certain embodiments, the lid is in direct contact with the culturing medium containing cells once loaded with the culturing medium and the cells.

### BRIEF DESCRIPTION OF THE DRAWINGS

This application contains at least one drawing executed in color. Copies of this application with color drawing(s) will be provided by the Office upon request and payment of the necessary fees.

FIGS. 1A-1K show that microcontainers enabled IrECM-free organoid culture. FIG. 1A shows production of microwells by photolithography and impression molding. FIG. 1B shows a novel microwell-based design enclosing each organoid within a “microcontainer” of culture media. FIG. 1C shows that seven thousand two hundred individually addressable organoids were grown in a single 24-well plate, with a series of two-fold magnifications. FIGS. 1D and 1E show that organoids grown in microcontainer format reached stable morphology within 14 days, by tracking individual organoids (FIG. 1D) and classifying the number of organoids with lumens (FIG. 1E), with each blue line showing the percent lumenization of a subgroup of 10 organoids (220 organoids total) and the red line showing the grand mean. FIG. 1F shows that organoids were able to survive in microcontainers for months. FIG. 1G is a schematic of graphics for FIGS. 1H and 1I. Each well is summarized by mean±standard error (95% confidence interval). FIG. 1H shows qualitative assessment of organoid morphology showing differences between microcontainers and traditional microwells. n=36 organoids/well. FIG. 1I shows that microcontainers permitted the growth of high-grade organoids in the absence of Matrigel. n=36 organoids/well. FIG. 1J is an immunostaining showing laminin α3 and collagen IV secretion from organoids within microcontainers. FIG. 1K shows gelation of microcontainer contents was visible upon removal from agarose. Scale bars are 50 mm.

FIGS. 2A-2G show that microcontainers enabled IrECM-free organoid culture. FIG. 2A is a microscopy showing the diffusion of different substances from microcontainers after 48 h culture. At left, AlexaFluor-594-labeled Matrigel stayed within microcontainers. At right, 15 nm diameter quantum dots diffused freely from the microcontainers. FIG. 2B shows the size distribution of organoids in microcontainers, with quadruplicate specimens and technical duplicates, as measured by cross-sectional area. FIG. 2C shows a microcontainer organoid with lumen, imaged with confocal microscopy. Scale bar is 50 μm. FIG. 2D shows the confusion matrix for random forest binary classifier used to distinguish lumenized from non-lumenized organoids. FIG. 2E shows the comparison of organoids in supra-gelling and sub-gelling matrices. Organoids were grown in microcontainers loaded with either Matrigel or collagen I at concentrations either below or above the threshold necessary for gelation. After 13 days, morphology was qualitatively assessed, as per the methodology in FIGS. 1H-1I. FIG. 2F shows the comparison of organoids in low-volume microwells, high-volume microwells, and high-volume microwells fed with conditioned media from low-volume microwells. After 5 days, morphology was qualitatively assessed, as per the methodology in FIGS. 1H-1I. FIG. 2G shows the staining matrix plugs for agarose content. Lugol's iodine stained agarose purple and left protein unstained. After dissecting microcontainers, bulk agarose stained purple, at left. At right, a cylindrical plug of matrix from a microcontainer did not stain purple except for the microcontainer lid.

FIGS. 3A-3D show that microcontainer organoids had generally accepted hallmarks of mammary organoids. FIG. 3A is an organoid lumenization shown via orthogonal projection. Arrowheads denote secondary chamber within lumen. FIG. 3B is a flow cytometry plot showing sorted luminal and myoepithelial populations. FIG. 3C is an immunostaining showing bilayered organization of K14+ and K18+ cells. Organoids in the top row used cells from a 66-year old specimen; organoids in the bottom row were from a different experiment using cells from a 19-year-old specimen. FIG. 3D is an immunostaining showing basal polarization of integrin. Zoomed inset at right. Scale bars are 50 μm.

FIGS. 4A-4B shows that microcontainer organoids exhibited generally accepted hallmarks of mammary organoids. FIG. 4A shows the lineage composition of various primacy mammary gland specimens, as measured by flow cytometry. CD133 marked luminal cells and CD271 marked myoepithelial cells. FIG. 4B shows line intensity profile showing bilayered stratification of luminal KRT18 and myoepithelial KRT14 from a confocal section of an immunofluorescently stained microcontainer-based HMEC organoid. Scale bar is 50 μm.

FIGS. 5A-51 show that mammary organoids grown in microcontainers exhibited contractility. FIG. 5A is filmstrips showing single contractions of three organoids. Maximum dilation and contraction were denoted with blue and red arrows, respectively. FIG. 5B is overnight dynamics of a single organoid showing 17 contraction events. Green highlighting shows a sequence of high-frequency contractions. FIG. 5C shows dynamics of a single contraction. FIG. 5D shows Poincaré plot embedding temporal dynamics for mammary organoids. Examples of heart, exponential, and normal distributions are shown for comparison to organoid contractions. Density in red. n=50 organoids. FIGS. 5E and 5F show contractility frequency (FIG. 5E) and magnitude (FIG. 5F) histograms for a population (n=57) of organoids across 21 hours. Kernel density in blue, accompanying box-and-whisker plots in red. FIG. 5G shows immunostaining for smooth-muscle actin, phalloidin, and cytokeratin 14. FIG. 5H shows that Latrunculin (2 μM) (n=20 organoids) and jasplakinolide (2 μM) (n=22 organoids) treatments, targeting actin, prevented contractions. FIG. 5I shows that ML-7 treatment, targeting smooth muscle myosin light chain kinase, prevented contractions. n=59 organoids. Scale bars are 50 μm.

FIGS. 6A-6B show that mammary organoids grown in microcontainers exhibited contractility. FIG. 6A shows that morphology of organoids in the presence or absence of prolactin demonstrated no obvious differences with prolactin treatment. FIG. 6B shows that in one, but only one, case, an organoid and its surroundings stained positive for Sudan Black, a lipophilic dye that stains, among other things, milk secretions. Bottom-left shows a close-up of the sudanophilic organoid, and bottom-right shows a non-sudanophilic organoid. Scale bars are 50 μm.

FIGS. 7A-7E show the durability of differentiated states. FIG. 7A shows that organoids started culture in microcontainers and were subsequently transferred into IrECM culture for a total combined culture time of 14 days. Afterwards, 24-hour time-lapse movies were taken, summarized via filmstrip (FIG. 7B). FIG. 7B is graphs showing how the cross-sectional area of selected organoids changes over time. FIG. 7C shows the spatial localization of cross-sectional changes, with regions of change highlighted in red. Organoids transferred at 2 d or 4 d show localized contractions (blue arrowheads), whereas organoids transferred at 12 d show global contractions. FIG. 7D shows that organoids transferred out of IrECM culture continued to show contractions. FIG. 7E shows that contractile behavior persisted in organoids cultured for 157 days. In FIGS. 7D and 7E, maximum dilation and contraction are denoted with blue and red arrowheads, respectively. Scale bars are 50 μm.

FIG. 8 shows that organoids cultured in microcontainers for at least several days retained contractility after being transferred to other culture systems. Alternate time scales for organoids in FIG. 7B microcontainer transfer experiment. An extended duration (600 m) is shown for the 6-hour organoid, and an abbreviated duration (200 m) is shown for the 12 d organoid.

FIGS. 9A-9D show the RNA-seq comparison of microcontainer organoids and primary cells. FIG. 9A shows the relative gene expression for characteristic genes of luminal cells, myoepithelial cells, and the extracellular matrix genes for standard two-dimensional culture, standard three-dimensional culture, microcontainer culture, and primary tissue specimens. FIG. 9B shows the principal component analysis of specimens on the basis of the genes listed in FIG. 9A, with specimens colored by culture method. FIG. 9C shows correlation analysis of lineage-specific genes across culture conditions, divided by lineage. Gene expression values were regularized and log transformed as per the DESeq2 r log function. Pearson coefficients are shown above. FIG. 9D is a lollipop plot showing the top ten genes whose expression levels most highly converged or diverged from that of primary tissue.

FIGS. 10A-10D show that gene expression of microcontainer organoids resembled gene expression of primary tissue. FIG. 10A shows relative gene expression for extended list of extracellular matrix genes. FIG. 10B shows the principal component analysis of culture conditions, divided by lineage. Genes with the highest loadings for each varimax-rotated component shown along each axis. FIG. 10C is a description of gene expression distance determination. FIG. 10D is a lollipop plot showing the top ten genes whose expression levels most highly converged or diverged from primary tissue, for microcontainers vs standard 3D culture.

### DETAILED DESCRIPTION

Disclosed herein is a method of producing an organoid without any exogenous extracellular matrix or in the presence of a concentration of exogenous extracellular matrix lower than its minimum gelling concentration. The method entails scaffold-free culturing cells specific for a desired organoid type in a confined volume such as a microcontainer. In certain embodiments, the confined volume is about 2-fold, about 3-fold, about 4-fold, about 5-fold, about 6-fold, about 7-fold, about 8-fold, or 9-fold, or about 10-fold of the average volume of the desired organoid. The microcontainer disclosed herein comprises walls composed of a hydrogel material and a hydrogel lid, wherein once cells and culturing medium are loaded in the microcontainer, the hydrogel walls and lid prevent the cells from escaping but allow air and liquid exchange with the environment, while slowing or stopping the diffusion of macromolecules. Various hydrogel materials such as agarose, gellan, alginate hydrogels or a combination thereof can be used for the walls and the lid of the microcontainer. In certain embodiments, the microcontainer has a diameter between about 100 μm and 150 μm. In certain embodiments, the microcontainer has a depth between about 100 μm and 350 μm. In certain embodiments, the microcontainer has a diameter of about 100 μm and a depth of about 200 μm. Although one skilled in the art can adjust the diameter and depth of the microcontainer to optimize the quality and/or quantity of the organoids, microcontainers that are too wide or too deep may be difficult to manipulate or fabricate, whereas microcontainers that are too shallow or too narrow may not hold a sufficient quantity of the cells.

More specifically, the method includes the steps of loading organ-specific cells in a microcontainer containing a culturing medium, overlaying a hydrogel over the culture containing the cells such that the hydrogel forms a lid in direct contact with the surface of the culture to seal the culture, and culturing the cells in the hydrogel-sealed microcontainer to obtain the organoid. The term “seal” as used herein in the context of sealing the microcontainer means preventing or substantially preventing escape or diffusion of the cells or macromolecular components such as extracellular matrix into the culture medium but allows air or fluid exchanges with the environment. Preferably, the macromolecular components accumulate near the cells to promote the growth of organoids. In certain embodiments, the cells include epithelial cells (e.g., mammary, prostate, intestine, sweat, lung, esophageal), fibroblast cells, stem and progenitor cells from embryonic or iPSC origin, neuroendocrine cells, immune cells, or a mixture or combination thereof. Mammary epithelial cells, or mammary stem and progenitor cells that give rise to the differentiated daughter epithelia, self-organize to form bilayered acini that constitute organoids, and sometimes these organoids elaborate still further into branching structures, and into organoids that exhibit high order differentiation such as contraction of the myoepithelial or milk production by the luminal epithelia. Epithelia from prostate and intestine are similar, and organoids from these tissues are known in the art. iPSC derived differentiated cells of multiple lineages are known to self-organize into organoid like structures, the precise lineages depend upon the differentiation protocols that are followed. Inclusion of immune cells with epithelial organoids, or fibroblasts with epithelial organoids, or adipo-stromal cells with epithelial organoids constitutes a means of reconstituting stromal elements of the organoid microenvironment that enables one to examine impacts of cell-cell communication between different compartments of a tissue. In certain embodiments, the culturing medium has a higher density than the hydrogel lid. For example, the culturing medium has a density between about 1.1 g/ml and about 1.2 g/ml and the hydrogel lid has a density of about 1.0 g/ml. In certain embodiments, the culturing medium comprises one or more biological colloid to achieve a higher density than the lid. Various biological colloids at various concentrations can be used to increase the density of the culturing medium. Some nonlimiting examples of biological colloids include dextrin, maltodextrin, albumin (e.g., bovine serum albumin or bovine serum albumin, fraction V), PEG-8000 and hydroxyethyl starch. In certain embodiments, the hydrogel for the lid comprises agarose, gellan, alginate hydrogels, or a combination thereof.

In another aspect, this disclosure relates to an organoid produced by the culturing methods disclosed herein. In certain embodiments, the organoid produced in vitro in the absence of ECM or in the presence of low concentration of ECM exhibits contractility. In certain embodiments, the organoid produced in vitro in the absence of ECM or in the presence of low concentration of ECM exhibits pulsatile contractility. In certain embodiments, the organoid produced in vitro in the absence of ECM or in the presence of low concentration of ECM expresses alpha-smooth muscle actin (ASMA).

Mammary epithelial organoids are traditionally grown in IrECM, which is typically derived from a non-human source, e.g. rodent Engelbreth-Holm-Swarm tumor cells (Hassell et al., 1980), or less often from a non-mammary human source (Okoh et al., 2013). It has been demonstrated that IrECM provides essential cues for maintaining proper organization and polarity in epithelial organoids and that culturing HMECs in suspension or low attachment cultures does not efficiently yield polarized acinar morphologies (Chanson et al., 2011). Previous work (Streuli and Bissell, 1990) has shown that mammary epithelial cells secrete matrix components under some culture conditions. As disclosed herein, a confined volume was able to concentrate these secreted components and allow establishment of polarity cues.

Cultured HMECs express extracellular matrix components (Stampfer et al, 1981, 1993), making it puzzling that exogenous scaffolds are necessary for HMEC organoids. One plausible explanation is that cells do not express sufficient quantities of matrix for their culture systems, as the concentration of matrix polymers must exceed a minimum threshold to gel into a network (Yurchenco et al., 1985). In systems such as hanging droplets (Djomehri et al., 2019) or ultra-low-attachment plates (Keller et al., 2019), matrix secretions are diluted into a relatively large volume of culture media, an issue present even in systems such as droplet microfluidics (Yu et al., 2010), and dilution may prevent this gelation threshold from being reached.

Disclosed herein is a novel microcontainer culture system that maximizes the concentration of the endogenous, secreted matrix. Within the microcontainers, mammary organoids can be reconstituted from HMECs in the absence of IrECM. Microcontainer culture produces multiple arrays of 103-104 individually addressable organoids, meeting or exceeding the throughput of state-of-the-art techniques such as micropocket culture (Zhao et al., 2019). Mammary organoids in microcontainers demonstrate self-organization, polarization, and functional differentiation, including pulsatile myoepithelial (MEP) contractility (Mroue et al., 2015), a physiological behavior not observed previously in reconstituted organoids.

Exogenous IrECM is generally regarded as necessary to sustain mammary epithelial organoids with normal apical-basal polarity and bilayered morphology. Microcontainers appear able to bypass this requirement, possibly due to allowing the secreted endogenous matrix to accumulate in a confined volume, allowing the matrix to provide microenvironment cues (Cerchiari et al., 2015; Chanson et al., 2011). Optimal conditions for mammary organoids in microcontainers include at least some exogenous matrix (0.5 mg/mL-1 mg/mL Matrigel), but Matrigel-free culture is viable to a much greater extent in microcontainers than in microwells. The profile of laminin genes expressed by organoids within microcontainers is distinct from the profile of laminins characterized in Matrigel, which suggests that the microcontainer microenvironment may provide cues that are elusive when using the exogenous matrix formulas typical to organoid culture.

MEP contractility is a key physiological function of mammary epithelia that is attainable via microcontainer culture. MEP cells, along with fibroblasts, can deform the matrix, such as by contracting collagen (Nielsen et al., 2003), and it has been shown that such contractions may be mediated through MEP motility (Buchmann et al., 2019). The pulsatility of contractions seen in microcontainers is novel. Relatedly, ASMA is a key clinical marker of MEP cells (Lategan, n.d.) whose expression is either entirely absent or rapidly declines (Taylor-Papadimitriou et al., 1989) during HMEC culture. Differentiation of MEP cells toward an ASMA-expressing phenotype is influenced by media composition (Fridriksdottir et al., 2017), and it is demonstrated herein that microcontainer culture has a similar differentiating effect. Achieving this level of differentiation may aid examination into the tumor-suppressive functions of MEP cells. Adding stromal tissue components and retaining hormone response genes, possibly through cyclic application of estrous hormones and inclusion of TGF-beta receptor inhibitors in the media, could significantly narrow the gap between organoid culture and primary tissue.

Microcontainer culture offers additional advantages other than an exogenous extracellular matrix-free culture. Microcontainer throughput is among the highest-throughput of organoid culture techniques, rivaling microwells (Cerchiari et al., 2014), micropockets (Zhao et al., 2019), and droplet microfluidics (Yu et al., 2010). The relative simplicity of the approach lends itself to scalability. Although matrix components can accumulate in microcontainers, it is likely that microcontainers are in paracrine contact with one another due to diffusion of low-molecular-weight factors through the agarose. Microcontainer culture is useful for longitudinal tracking of organoids, due to microcontainers keeping organoids in defined locations for months. Pulsatile contractility, which stands out as a functional behavior, appears to be either inhibited, absent, or unobservable in other approaches. Taken together, microcontainer culture provides the means to study statistically robust quantities of physiologically relevant organoids without the cost or confoundment of IrECM.

A prospect for microcontainers is producing microenvironments in vitro that cannot be produced using available exogenous scaffolds. The extracellular matrix consists of a wide variety of proteins, including at least twenty-eight distinct collagens (Ricard-Blum, 2011) and at least fourteen distinct laminins that are found in different combinations in different tissues. The scope of matrices commercially available is far narrower. However, microcontainers may be able to produce usable microenvironments from any combination of matrix proteins secreted by cells. In the case of HMECs, cells produce a microenvironment enriched in laminin-332, as opposed to the laminin-111 characteristic of Matrigel. In this manner, microcontainers may provide access to a broader range of more physiological microenvironments than otherwise attainable.

It is not trivial that cells would be capable of reconstructing their native microenvironments. The notion presupposes that cells retain lineage-specific information about their native matrix composition, presumably via epigenetic states; previous work has shown that cultured primary HMECs retain lineage-specific DNA methylation patterns consistent with uncultured tissue for at least four passages (Miyano et al., 2017). Curiously, uncultured HMECs show less expression of the laminin-332 genes than do cultured HMECs. From the perspective of dynamic reciprocity (Roskelley and Bissell, 1995), one could reason that cells produce less matrix as the abundance of native matrix increases. Or viewing cell culture as a wound healing response, perhaps cells “repair” their microenvironment by producing their native matrix. The cells may retain the necessary information to reconstruct tissue-appropriate microenvironments within their epigenetic memories.

Extracellular matrix mechanics are of particular interest in the mammary gland (Chaudhuri et al., 2014; Pelissier et al., 2014; Schedin and Keely, 2011). Agarose mechanically isolates microcontainers from one another, as well as from the plastic cultureware, which should limit the stiffness experienced by the organoids.

Although the working examples illustrate mammary epithelial cells, microcontainer culture is useful for culturing other cell types. Microcontainers permit the buildup of endogenous secreted matrix, which is useful for cells that rely on IrECM for in vitro culture, including the epithelium such as the prostate and gut or liver hepatocytes. Furthermore, stem and progenitor cells, which are exquisitely sensitive to their microenvironment, would benefit from the endogenous matrix. In circumstances where cells of interest cannot secrete sufficient matrix on their own, cell types can be combined, for example, stromal cells can be combined with epithelial cells. By increasing the availability of tissue-specific, species-specific microenvironments, microcontainer culture may increase the physiological validity of 3D cell culture.

As demonstrated in the working examples, mammary organoids produced by the disclosed methods exhibited functional differentiation, specifically contractility, and were composed of human cells within autologous extracellular matrix. These results suggest that human mammary epithelial cells secrete sufficient matrix proteins to sustain their own microenvironment, bypassing the need for using exogenous matrices.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Materials and Methods

Small molecules: Latrunculin B was purchased from Enzo (cat #BMLT110-0001, lot #8221661). Jasplakinolide was purchased from Enzo (cat #ALX-350-275-0050, lot #120091480). ML-7 was purchased from Sigma (cat #12764-5MG, lot #SLBX6943). RepSox was purchased from Sigma (cat #R0158-5MG). SB431542 was purchased from Sigma (cat #54317-5MG).

Cell culture: Finite-lifespan HMECs were provided by the Human Mammary Epithelial Cell (HMEC) Bank (Stampfer and Garbe, n.d. For standard 2D culture, primary human mammary epithelial cells at passage 4 were established and maintained in M87A medium as previously described (Garbe et al., 2009). Mycoplasma testing was performed prior to all experiments in this study.

Antibodies and stains: A comprehensive list of antibodies and stains is in Table 1 below.

TABLE 1
Antibodies and Stains

| Antibody | Product | Application |
|---|---|---|
| anti-human keratin 19 | Biolegend 628502 (clone A53-B/A2) | IF (1:1000) |
| anti-human keratin 14 | BioLegend 905301 RB-9020-P (clone Poly9053) | IF (1:1000) |
| anti-human keratin 18 | Novus NBP1-97714 | IF (1:1000) |
| anti-human CD104 | Chemicon MAB1964 (clone 3E1) | IF (1:1000) |
| anti-human laminin, alpha-3 | R&D Systems MAB21441 (clone 546215) | IF (1:1000) |
| anti-human collagen IV | Sigma C1926 (clone COL-94) | IF (1:1000) |
| anti α-smooth-muscle actin | Sigma A2547 (clone 1A4) | IF (1:1000) |
| anti-human estrogen receptor | abcam ab16660 | IF (1:333) |
| QTRACKER Qdot vascular labels | Thermo Q21061MP | live (2 μM) |
| Alexa Fluor 594 NHS ester | Thermo A20004 | conjugation |
| phalloidin iFluor-647 | abcam ab176759 | FC (1:500) |
| Hoechst 33342 | Thomas Scientific C979U06 | FC (1:500) |
| anti-human CD271, Cy5.5 conjugate | BioLegend 345112 (clone ME20.4) | FC (1:100) |
| anti-human CD133, PE conjugate | Miltenyl 130-080-801 (clone AC133) | FC (1:50) |

Immunofluorescence: Organoids were processed for immunofluorescence while still within microcontainers. All samples were fixed with 4% formaldehyde for 20 minutes and incubated in blocking buffer (10% heat-inactivated goat serum in PBS+0.5% Triton X-100) at 4° C. for at least 1 day. Primary antibodies were diluted in blocking buffer and added to the sample. After at least 1 day incubating at 4° C. with the primary antibodies, samples were washed several times with PBS+Triton X-100 for at least 1 day and incubated with fluorophore-conjugated secondary antibodies diluted at a concentration of 1:200 in blocking buffer for approximately 1 day. All samples were washed with PBS+1 μg ml⁻¹ DAPI for at least 1 hour before imaging. For widefield imaging, whole organoids were imaged in situ within the microcontainers. For confocal imaging, the agarose lids of the microcontainers were scraped away with a silicone cell scraper, then replaced with a glass coverslip in order to reduce the working distance from the microscope objective.

Flow cytometry: Each sample was transferred to a collection tube and resuspended in PBS. Fluorescently-tagged antibodies were added at concentrations shown in Table 1 and incubated for 30 minutes on ice. Labeled cells were washed three times with PBS to remove unbound antibody and resuspended in flow buffer (PBS with 2% BSA, 1 mM EDTA, and 1 μg/mL DAPI). Cells were sorted on a BD FacsAria III. LEPs were defined as CD133+/CD10− cells and MEPs were defined as CD133−/CD10+ cells, with DAPI+ cells discarded.

Image acquisition: All confocal microscopy images were acquired using a Zeiss LSM 880 with Airyscan running Zeiss Zen Software. Subsequent deconvolution was performed with AutoQuant. All brightfield microscopy images were acquired using a Nikon Eclipse Ti-E with stage-top incubation and high-speed electro-magnetic stage with piezo Z, running Nikon Elements software. Subsequent workup and image analysis were performed using ImageJ.

Photolithography: Freestanding SU-8 features on silicon wafers were fabricated using standard photolithographic techniques. All recipes used for photopatterning were adapted from MicroChem's technical specification sheets. To obtain cylindrical microwells of 100 μm diameter and 200 μm depth, 60 grams of SU-8 2075 (MicroChem) was spun on a 125 mm technical-grade silicon wafer (UniversityWafer) at 300 rpm for 30 seconds followed by accelerating at 100 rpm/s to a final speed of 700 rpm for 30 seconds. The wafer was soft-baked for 20 minutes at 95° C., UV-exposed with a 1700 mA 365 nm LED source (ThorLabs) at full power in contact mode for 15 minutes through a photo-mask designed in Adobe Illustrator and purchased from Outputcity Co., post-exposure baked for 5 minutes at 95° C., and developed in SU8 developer (MicroChem) for 20 minutes. The patterned substrate was washed with isopropanol/water and baked at 95° C. for 20 minutes. The silicon master was taped to the bottom of a 15 cm Petri dish and potted with Sylgard 184 (Dow Corning). After curing at 65° C. overnight, the molded elastomer was peeled off the wafer and inspected by microscopy to assess the diameter and depth of the lithographic features. The wafer was rendered hydrophobic by treatment with SigmaCote (Sigma-Aldrich) and subsequent isopropanol washes. At this point, the wafer was ready for production of agarose microwells.

Microcontainer production: Photolithographic masters, prepared as described above, were sanitized with 70% ethanol and kept at 65° C. until use. 20% 5.5 dextrose-equivalent maltodextrin was prepared in 2×PBS and dissolved with gentle heating before 0.2 μm filtration. A solution of 3% agarose in diH₂O was autoclaved and mixed 1:1 with the warm, filtered maltodextrin solution. 15 mL of this mixture was immediately dispensed onto a photolithographic master and allowed to gel at 4° C. Demolding resulted in agarose microwells. Microwells were equilibrated with cell culture media over two days. Next, HMECs were loaded into microwells by panning and sedimentation. Microwells were inspected by microscopy to confirm cell loading, washed once with cell culture media to remove excess cells, aspirated to near-dryness, and then incubated with 10% 5.5 dextrose-equivalent maltodextrin in cell culture media for 20 minutes at 37° C. Microwells were again aspirated to near-dryness before being overlaid with 1.0% ultra-low-melting agarose in cell culture media and brought to 4° C. to gelation. Upon gelation of the overlaid agarose, the resulting products are microcontainers. HMEC organoids in microcontainers have their media changed 24 hours after microcontainer formation and once a week thereafter.

Organoid harvesting and RNA isolation: Organoids were harvested from microcontainers prior to RNA isolation. First, the microcontainers were incubated with collagenase for two hours at 37° C. to dissolve any gelled matrix within the microcontainers that would prevent release of the organoids. Next, the microcontainers were inverted with a lab spatula, in order to expose the agarose lids that were otherwise pressed against the tissue culture plastic. Next, the agarose lids of the microcontainers were removed with a silicone cell scraper, exposing the organoids within the opened microcontainers. Next, the tip of a P1000 micropipette was cut to a 1 mm diameter, and PBS was added to a depth of 2 mm over the opened microcontainers. By gently and repeatedly pipetting up and down, the organoids were aspirated out of their microcontainers and transferred to a collection tube. Retrieval of the organoids and corresponding emptying of the microcontainers were verified by microscopy. Approximately 9600 organoids were pooled for each specimen to be analyzed. The retrieved organoids were dispersed into a single-cell suspension with 0.25% trypsin-EDTA and strained through a 40 μm filter before being LEP and MEP lineages were separated with fluorescence-activated cell sorting, as disclosed above. Total RNA from FACS-sorted cells were isolated using Quick DNA/RNA microprep plus kit (Zymo Research). RNA was submitted to the City of Hope Integrative Genomics Core Facility for library preparation and sequencing.

Image acquisition: All confocal microscopy images were acquired using a scanning confocal microscope (Zeiss LSM 700 running Zeiss Zen software) and deconvolved with AutoQuant. All other microscopy images were acquired using an inverted motorized microscope equipped with live-cell incubation (Nikon Ti-E running Nikon Elements software).

Contraction temporal analysis: Organoid contractions were quantified either by hand or by brightness change analysis, as indicated. For brightness change analysis, time-lapse movies were taken, and the time-derivative of the whole-field image brightness was calculated. Local maxima in the time-derivative were interpreted as contractions and spot-checked by eye.

RNA-seq: RNA library preparation was done with either the KAPA mRNA Hyper kit (cat #KK8581) or the Takara SMART-Seq v4 Ultra Low Input RNA kit (cat #634888). Sequencing was done on an Illumina HiSeq 2500. Reads were aligned to Homo sapiens reference genome hg19 using TopHat2. Unless noted otherwise, exploratory, visualization, and differential gene expression analysis was carried out in R. Briefly, for heat maps, raw counts were normalized with the trimmed-means-of-means method then converted to counts-per-million units. For Pearson correlation analysis, any genes with fewer than 100 reads across all specimens were discarded, then DESeq was used with default settings to identify differentially expressed genes to include in the analysis. Principal component and geometric mean analyses were performed on the entire set of genes. PANTHER inquiries were submitted to www.pantherdb.org.

Heart rate variability analysis: Heart rate data was collected with a Polar H7 Bluetooth Heart Rate Sensor using the first author's heart.

Lumenization analysis: Two independent methods were used to assess luemnization. First, organoids were fixed in 4% formaldehyde, stained with phalloidin (for actin) and Hoechst 33342 (for nuclei), and imaged by confocal microscopy. A nucleus-free cavity ringed by phalloidin in the center of an organoid confirmed the presence of a lumen. Second, organoids were classified as lumenized or non-lumenized using a random forest classifier implemented in CellProfiler Analyst. A training set comprising 5% of the image data was manually curated, classifying organoid images as lumenized, non-lumenized, or unclassifiable. Organoid images had a battery of measurements performed on them, which were used as parameters for the random forest classifier. After training, the classifier evaluated all organoids for lumenization at all time points available.

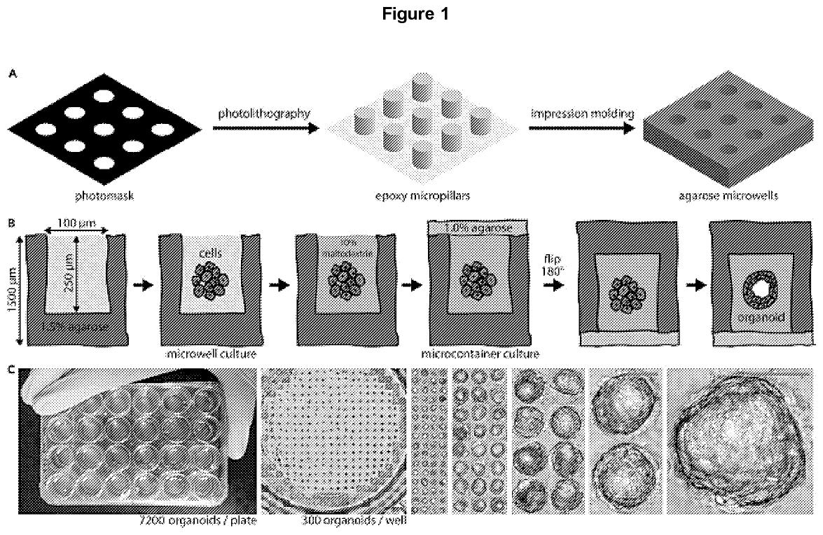

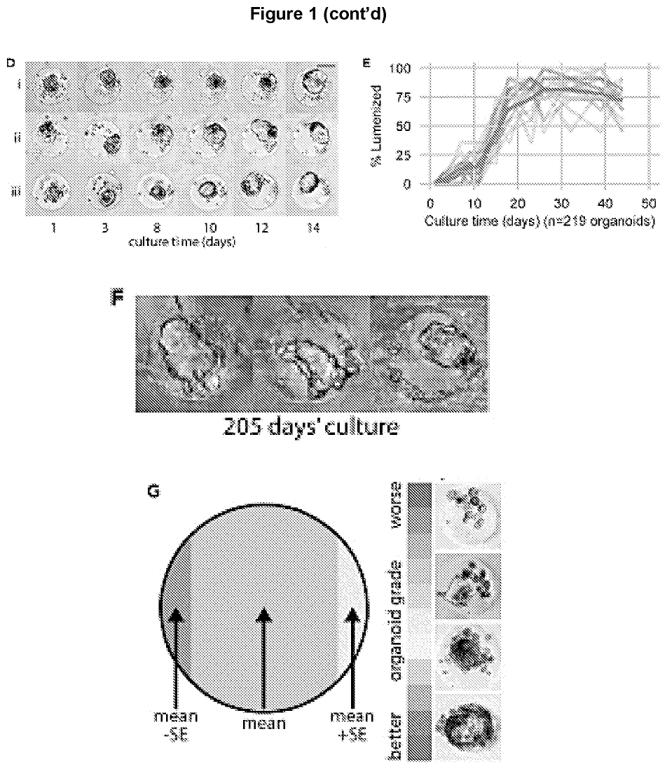

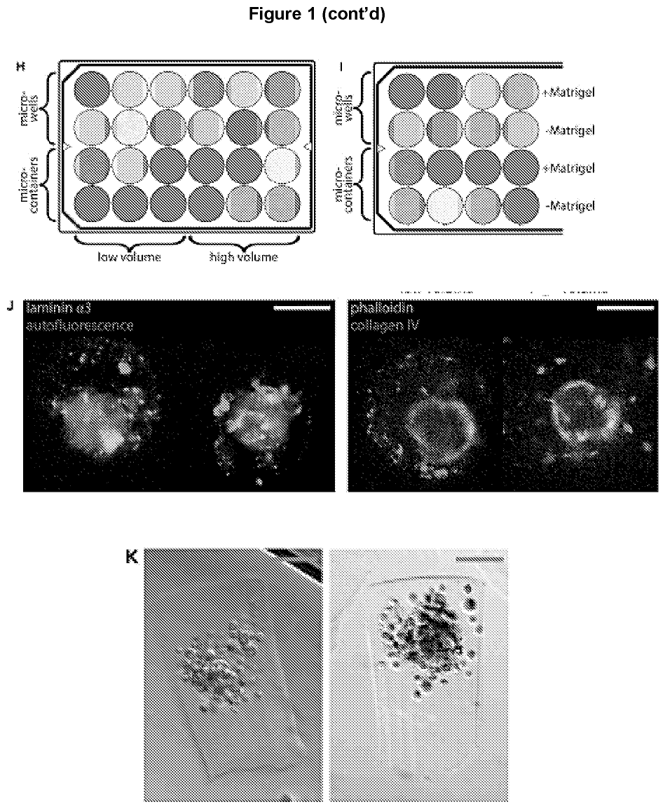

Statistical analysis: For analysis in FIGS. 1G-1H, 95% confidence intervals (CI) were calculated based on sample standard error (SE) with a finite population correction (FPC). FPC=sqrt(N−n)/(N−1)), where N is the number of organoids in the well (N=300) and n is the number of organoids observed (n=36). Then, SE=sqrt(p*(1−p)/n)*FPC, where p is the fraction of “good” organoids. The margin of error (MOE), where MOE=1.96*SE, where 1.96 is the critical value constant for a 95% confidence interval. Finally, the 95% CI was calculated as CI=sample mean+/−MOE.

### Example 1: Production of Organoids Using Microcontainers

This example demonstrates successful production of organoids without any exogenous extracellular matrix using microcontainers.

A microcontainer is a microwell made with standard photolithography techniques (FIG. 1A) that, after being loaded with cells, is sealed shut with a hydrogel lid (FIG. 1B), producing an enclosed liquid chamber instead of an open-topped microwell. Typically, flowing hydrogel across microwells would cause displacement of the microwell contents, rendering a lid strategy infeasible. To form the lids, microwells were first filled with a buffer brought to a high density (1.1 g/mL) with a soluble biocompatible solute such as maltodextrin or albumin (Table 2).

TABLE 2
High-Density Solutes Tested

| | |
|---|---|
| within microwell | layered over microwell |

Colloids Tested

| | | |
|---|---|---|
| 10.0% 4-7 dextrose-equivalent maltodextrin | 1.0% IX-A agarose | |
| 15.0% 4-7 dextrose-equivalent maltodextrin | 1.0% IX-A agarose | |
| 10.0% 9-13 dextrose-equivalent maltodextrin | 1.0% IX-A agarose | poor cell viability |
| 0.0% lipid-rich bovine serum albumin | 1.5% IX-A agarose | microwells evacuated of cells |
| 7.5% lipid-rich bovine serum albumin | 1.5% IX-A agarose | “donuting” in microwells |
| 10.0% lipid-rich bovine serum albumin | 1.0% IX-A agarose | |
| 10.0% lipid-rich bovine serum albumin | 2.0% IX-A agarose | “donuting” in microwells |
| 10.0% lipid-rich bovine serum albumin | 3.0% IX-A agarose | microwells evacuated of cells |
| 12.5% lipid-rich bovine serum albumin | 1.0% IX-A agarose | |
| 15.0% lipid-rich bovine serum albumin | 1.0% IX-A agarose | |
| 20.0% lipid-rich bovine serum albumin | 1.0% IX-A agarose | challenging to prepare |

Colloids Expected to Work

| | | |
|---|---|---|
| 10.0% bovine serum albumin, fraction V | 1.0% IX-A agarose | |
| 10.0% PEG-8000 | 1.0% IX-A agarose | possible phase separation |
| 10.0% dextran | 1.0% IX-A agarose | |
| 10.0% hydroxyethyl starch | 1.0% IX-A agarose | |

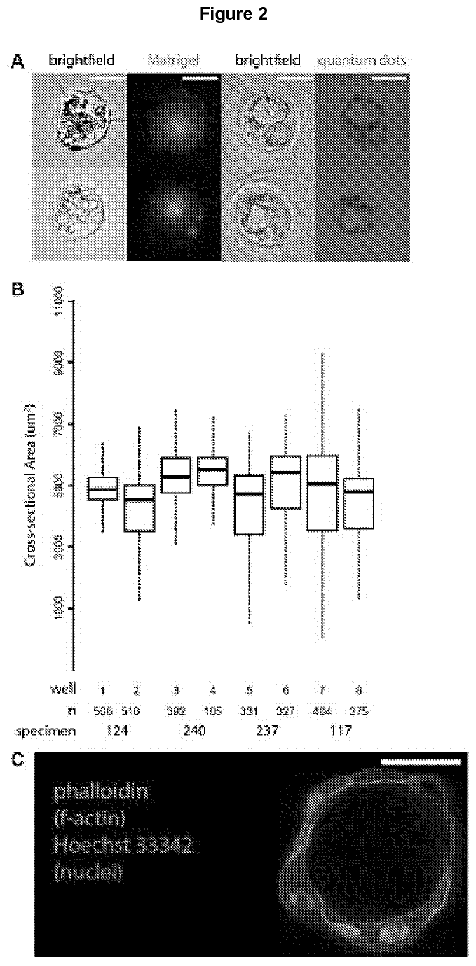

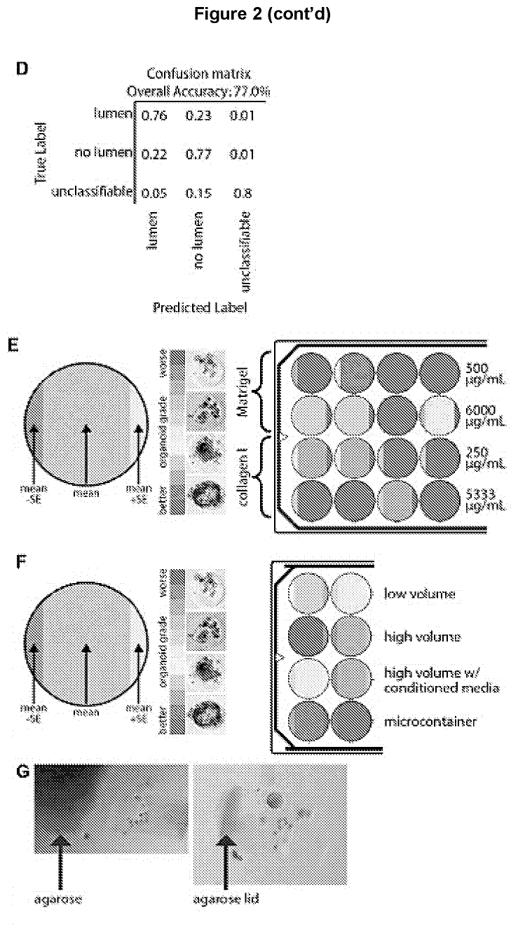

Next, the lower density (1.0 g/mL) lid hydrogel was flowed over the tops of the microwells and allowed to gel, sealing the microwells. Buoyancy prevented the lid hydrogel from flowing into the microwells, forming sealed pockets of dilute solution (i.e., microcontainers) underneath the gelled lid. A cylindrical microcontainer with 100 μm diameter and 200 μm depth confined a population of 20-100 HMECs to a 1.6 nL volume. A hydrogel lid composed of 1% agarose has an expected pore size on the order of 100 nm (Righetti et al., 1981), slowing the diffusion of proteins (Boyer and Hsu, 1992) from the microcontainer and preventing the escape of larger macromolecular aggregates. 15-nm quantum dots loaded into microcontainers to freely diffuse out, whereas fluorescently labeled Matrigel cannot (FIG. 2A).

Various types of hydrogels can be used to form the hydrogel lid, with some examples listed in Table 3 below.

TABLE 3
Hydrogels Tested

1.5% electrophoresis-grade agarose
2.0% electrophoresis-grade agarose
1.5% electrophoresis-grade agarose + 10% 4-7 dextrose-equivalent maltodextrin
1.5% electrophoresis-grade agarose + 15% 4-7 dextrose-equivalent maltodextrin
1.5% electrophoresis-grade agarose + 10% 4-7 dextrose-equivalent maltodextrin
1.0% sodium alginate + 1.0% finely dispersed calcium carbonate
1.0% Gelrite (gellan gum) + 10% 4-7 dextrose-equivalent maltodextrin

Other Hydrogels Can Be Used

1.0%-3.0% agarose + 0-15% maltodextrin
1.0%-2.0% sodium alginate + any calcium source
1.0%-3.0% gellan, guar, xanthan, or other viscous gums + 1-15% maltodextrin
10-30% gelatin, with or without enzymatic crosslinking
Any hydrogels made from acrylamide, bis-acrylamide, and/or derivatized polyethylene

Arrays of microcontainers provided a throughput of as many as 7200 organoids in a 24-well plate with microcontainers spaced on a grid of 500-mm pitch (FIG. 1C), yielding about 300 organoids within each of the 24 wells. On a grid, each organoid is individually addressable by automated microscopy, enabling each organoid to be tracked over months. With this sample size, a Student's t-test was able to evaluate an effect size of 0.1 at the 0.005 significance level with power >0.9, and a 24-category ANOVA was able to evaluate an effect size of 0.1 at the 0.05 significance level with power >0.9 (Table 4).

TABLE 4
Power analysis for organoids from a single plate
power analysis, t-test

| sample size | effect size | significance | power |
|---|---|---|---|
| n | (Cohen's d) | α | 1-β |
| 300 | 0.4 | 0.05 | 0.998 |
| 300 | 0.4 | 0.005 | 0.981 |
| 3600 | 0.1 | 0.05 | 0.989 |
| 3600 | 0.1 | 0.005 | 0.924 |

power analysis, ANOVA

| | sample size | effect size | significance | power |
|---|---|---|---|---|
| groups | n | f | α | 1-β |
| 24 | 300 | 0.09 | 0.01 | 0.997 |
| 12 | 600 | 0.08 | 0.01 | 0.996 |
| 6 | 1200 | 0.07 | 0.01 | 0.993 |
| 4 | 1800 | 0.06 | 0.01 | 0.975 |

Numbers assume a single 24-well plate of microcontainer organoids.

Mammary organoid morphology developed across a two-week period. Each microcontainer was initially loaded with 20-100 individual HMECs. Within 12-48 hours, these HMECs agglomerated into a single spheroidal mass. The exact size of the spheroids depended on how many HMECs were loaded, but 100 μm diameter microcontainers readily yielded spheroids with a cross-sectional area of about 4,800 μm² (about 78 μm diameter) (FIG. 2B). Self-organization of luminal epithelial (LEP) and MEP cells formed a bilayered structure, and, over the ensuing days, a lumen formed and grew within the organoid, as measured by confocal microscopy (FIG. 2C). Tracking individual organoids showed progressive lumen formation that typically reached maximum size by 14 days of culture (FIG. 1D). Applying binary classification to brightfield images of organoids allowed the percentage of organoids with lumen to be non-destructively measured at each time point (FIG. 1E) with reasonable sensitivity and precision (FIG. 2D). Organoids were able to be maintained with stable lumens for many months in microcontainer formats (FIG. 1F).

Initially, it was observed that organoids with healthy morphology were able to be obtained in microcontainers containing Matrigel or collagen I at concentrations either above or below their respective gelation thresholds (FIG. 2E).

This was notable, as hydrogels at concentrations below their gelation threshold did not provide a substrate for cell anchorage. Next, microcontainer cultures were compared to standard microwell culture (Napolitano et al., 2007) by assessing organoid morphology in both formats. The metric for organoid morphology was the fraction of non-squamous organoids with lumens, as assessed by brightfield microscopy (FIG. 1G). The organoids were cultured for one week in media supplemented with 1 mg/mL Matrigel, a concentration below Matrigel's gelation threshold (about 3 mg/mL) (Corning, n.d.) and below the 3-10 mg/mL typically used for hydrogel-embedded organoids (Corning, n.d.). Under these culture conditions, organoids grown in microcontainers showed a greater fraction of non-squamous organoids with lumens than organoids grown in microwells (FIG. 1H). This effect may have been due to microcontainers limiting the dilution of survival-promoting factors such as growth factors or matrix components into the culture media reservoir. Microcontainers and microwells were assessed across two media volumes: either the highest volume of culture media permissible by plasticware geometry (about 1500 μL) or the lowest volume of culture media that would not desiccate the microwells (200 μL) (FIG. 1H). More non-squamous organoids with lumens were observed in low-media microwells, consistent with the dilution hypothesis. In a follow-up experiment, microwells with high or low media volumes were compared to a third condition with high media volume provided by conditioned media from low media volume microwells. Conditioned high-volume microwells performed intermediate between high- and low-volume microwells, also consistent with the dilution hypothesis (FIG. 2F).

The organoid-culturing experiments were repeated but this time entirely omitting Matrigel from the culture media. After seven days of culture, non-squamous organoids with lumens were present in microcontainers, even in the absence of Matrigel (FIG. 1I). The fraction of non-squamous organoids with lumens was higher when 1 mg/mL Matrigel was present (mean=97%) than when no Matrigel was present (mean=73%), which suggests that although Matrigel-free conditions are feasible, the presence of Matrigel does provide some benefit to the quality of the culture. Conventionally, according to the manufacturer's instructions, Matrigel is used undiluted, corresponding to a concentration of 8-10 mg/mL, depending on the lot. Sometimes Matrigel is diluted down to its minimum gelling concentration, which is about 4 mg/mL. Below this concentration, Matrigel cannot form a gel and is not useful on its own to produce a microenvironmental scaffold. In microcontainers, Matrigel-free culture is feasible, but improved viability and lumenization of human mammary epithelial cells were observed when Matrigel is added to a concentration of 0.5-1.0 mg/mL.

Under Matrigel-free conditions, human extracellular matrix proteins were detected within microcontainers occupied by HMEC organoids. Immunofluorescence microscopy of whole microcontainers showed both laminin α3 and collagen IV surrounding organoids (FIG. 1J). The fluorescence signal was only visible within the microcontainers, with no signal in the agarose surrounding the microcontainers. This could be explained by laminin and collagen forming macromolecular aggregates sufficiently large to retard diffusion out of the microcontainers, consistent with their known polymerization behavior (Yurchenco et al., 1985). The presence of laminin al (Hansen et al., 2009) and laminin α3 was notable because they are key components of the mammary basement membrane. However, Matrigel does not contain laminin α3, only laminin al (Giannelli et al., 1999). The presence of laminin α3 could be explained by its production by the HMECs in microcontainers. The quantity of secreted protein was substantial: a plug of hydrogel that was visible by brightfield was able to be removed when a microcontainer was pried open (FIG. 1K). These plugs stained negative with Lugol's iodine (FIG. 2G), suggesting that they were not composed of the agarose used to construct the microcontainers. Furthermore, the plugs dissolved under collagenase treatment, suggesting that the matrix protein substantially comprised them.

### Example 2: Characterization of HMEC Organoids Grown in Microcontainers

This example verifies that HMEC organoids grown in microcontainers without IrECM conform to generally accepted standards of mammary organoid structure (Lategan, n.d.).

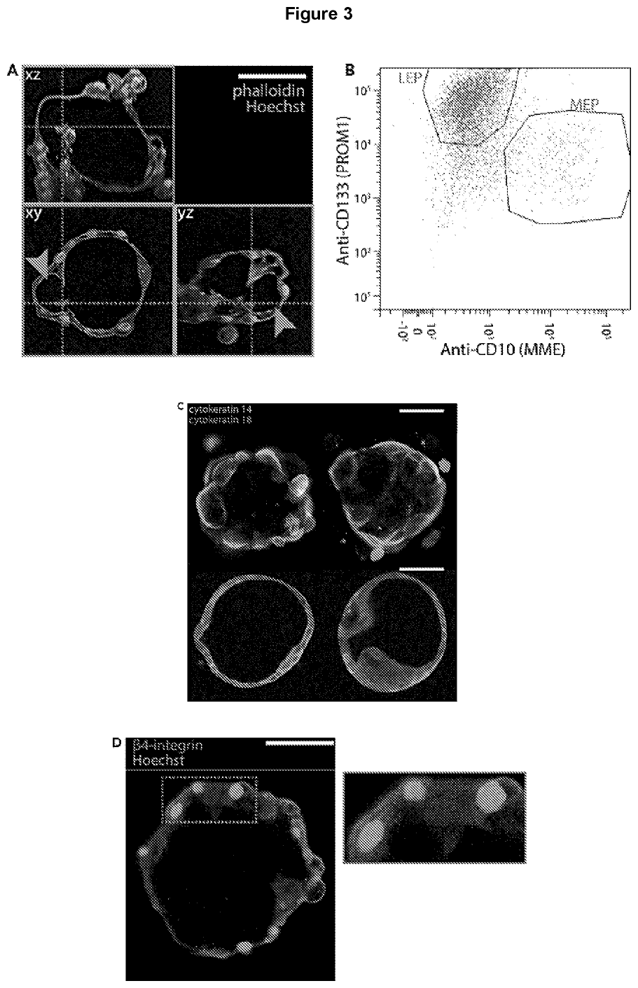

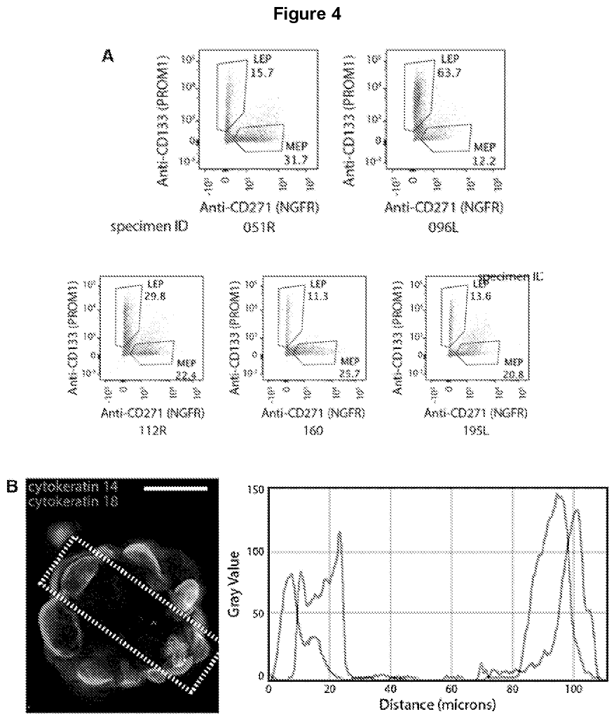

First, the microcontainer organoids reliably showed lumens by 14 days of culture, apparent by confocal microscopy (FIG. 3A). LEP and MEP cells were verified by both flow cytometry using antibodies against lineage-specific surface markers (CD133 for LEP cells and CD10 for MEP cells) (FIG. 3B) and immunostaining using antibodies against lineage-specific cytokeratins (KRT18 for LEP cells and KRT14 for MEP cells) (FIG. 3C). The abundance of MEP cells was apparently somewhat depleted relative to primary specimens (FIG. 4A), but this depletion was not evident when staining for cytokeratins in intact organoids and may be associated with the difficulty dissociating MEP cells from organoids for flow analysis. Confocal sectioning of immunostained organoids showed self-organization of LEP cells to the apical layer and MEP cells to the basal layer (FIGS. 3C and 4B), and immunostaining for integrin β4 showed basal polarization (FIG. 3D), as expected for mammary organoids. The establishment and maintenance of normal polarity and multi-lineage bilayers generally requires the extracellular matrix, indicating that HMEC organoids in microcontainers secrete their own basement membrane components.

### Example 3: High-Level Functional Differentiation of Myoepithelial Cells

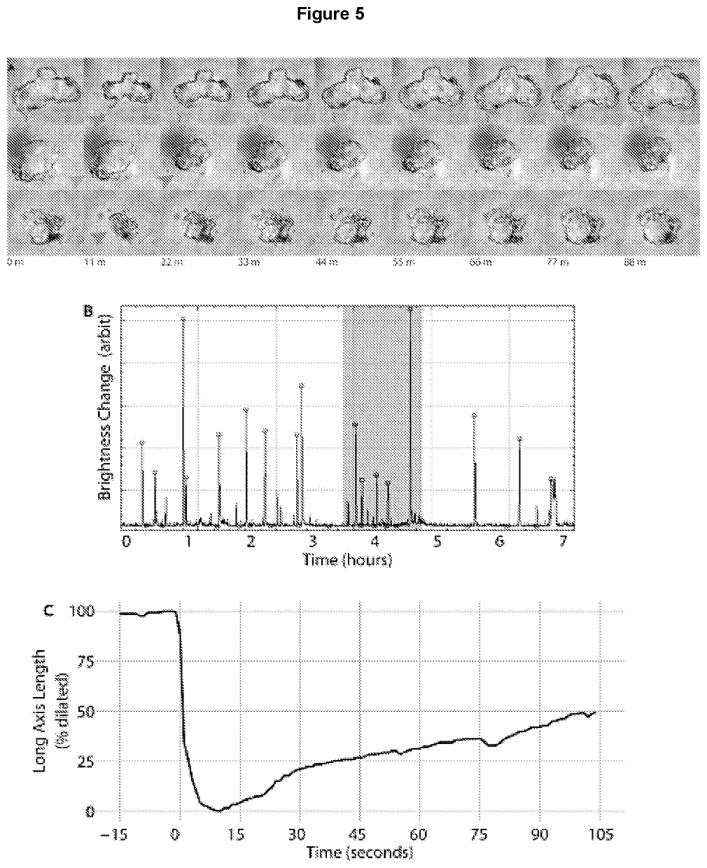

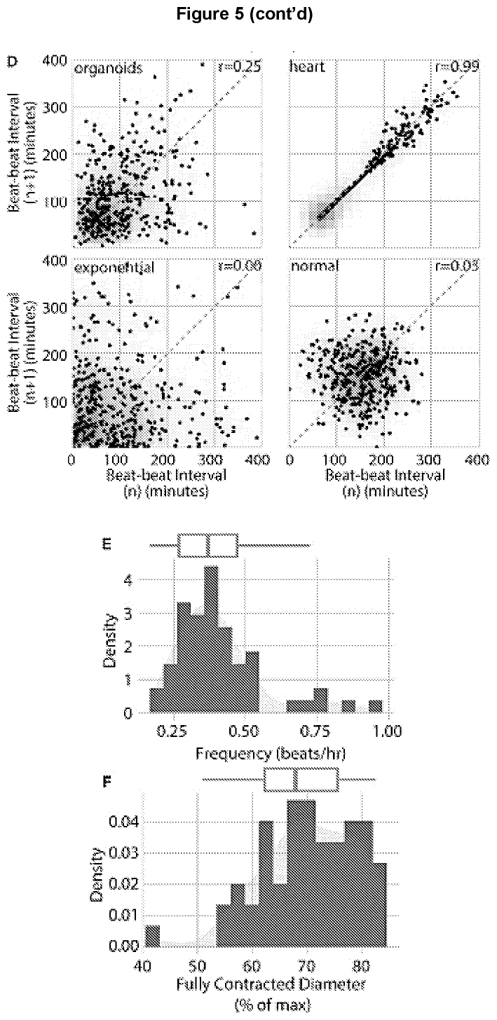

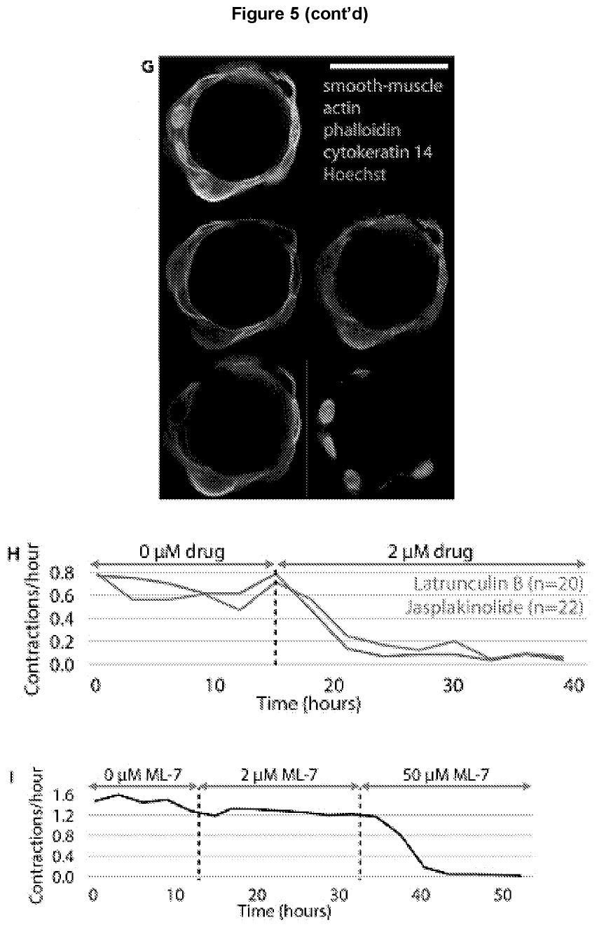

Surprisingly, the organoids obtained from microcontainers exhibited contractility. Contractility was not observed from any organoids grown in lid-less microwells that were otherwise substantially similar to microcontainers, suggesting that the constrained volume of the microcontainer provided by the hydrogel lid is necessary for contractility to occur. Although contractility is a known functional behavior of mammary tissue and has been observed in mouse mammary explants (Mroue et al., 2015; Sumbal et al., 2020), it has not been observed in reconstituted mammary organoids. Time-lapse microscopy showed that organoids in microcontainers gradually dilated across a span of one or more hours and then rapidly contracted (FIG. 5A). The dynamics of contractility varied by specimen and by experiment, but contractions typically had a frequency of about 0.1/hr (FIG. 5B), with individual contractions each spanning about 10 s (FIG. 5C). It was found that the frequency of successive contractions was often correlated, as shown in FIG. 5D via Poincare plot. Poincare plots, often used in cardiology, characterize the dynamics of pulsatile systems (Guzik et al., 2006). The temporal correlation is shown as a Pearson correlation. In the absence of temporal correlation, data points had a wide distribution and a near-zero Pearson correlation, as shown in the exponential and normal plots using simulated data. In the presence of a periodic rhythm, data points were centered elliptically on the identity line and had a high Pearson correlation, as shown in the cardiac plot. Mammary organoids showed a correlation of r=0.25, suggesting a temporally correlated but non-periodic contraction pattern. One possible explanation for such a pattern would be pulse trains (e.g., muscle clonus (Hidler and Rymer, 2000)), which indeed was observed in organoid contractions, such as the highlighted region of FIG. 5B. Physical fluidic models of lumen contractility have been proposed (Duclutet al., 2019; Ruiz-Herrero et al., 2017) but are not sufficient to predict pulse train or other temporally correlated behavior, suggesting that the contractility phenomenon is biomechanical.

Organoid contractions exhibit high variation in frequency and magnitude, which was assessed via time-lapse microscopy on a set of 57 organoids across 48 hours (FIG. 5E). Contraction magnitude was assessed by measuring the percent change of the long axis of organoids immediately before and after contractions (FIG. 5F). Various intervention attempts were made to alter the contraction kinetics, including withdrawing cholera toxin (an activator of protein kinase A) and isoproterenol (an adrenoreceptor agonist) from the media, as well as changing the microenvironment by adding collagen I at 0 to 2 mg/mL, low-molecular-weight hyaluronan at 0 to 2 mg/mL, and Matrigel at 0 to 4 mg/mL. In all cases, no effect was observed (Table 5).

TABLE 5
Matrix Compositions Tested
Matrix Compositions

| Matrigel | collagen I | 750 kDa HA | 2000 kDA HA | fibronectin | IX-A agarose | |
|---|---|---|---|---|---|---|
| μg/mL | μg/mL | μg/mL | μg/mL | μg/mL | μg/mL | |
| 0 | 0 | 0 | 0 | 0 | 0 | |
| 0 | 250 | 0 | 0 | 0 | 0 | gel doesn't flow into microwells |
| 0 | 5333 | 0 | 0 | 0 | 0 | |
| 200 | 0 | 0 | 0 | 0 | 0 | |
| 500 | 0 | 0 | 0 | 0 | 0 | |
| 750 | 0 | 0 | 0 | 0 | 0 | |
| 1000 | 0 | 0 | 0 | 0 | 0 | |
| 1000 | 500 | 0 | 0 | 0 | 0 | |
| 1000 | 500 | 200 | 0 | 0 | 0 | |
| 1000 | 0 | 200 | 0 | 0 | 0 | |
| 1000 | 0 | 0 | 300 | 0 | 0 | |
| 1000 | 0 | 0 | 1000 | 0 | 0 | |
| 1000 | 0 | 0 | 3000 | 0 | 0 | gel doesn't flow into microwells |
| 1000 | 0 | 0 | 0 | 23 | 0 | |
| 1000 | 0 | 0 | 0 | 0 | 100 | |
| 1000 | 0 | 0 | 0 | 0 | 200 | cells don't cohere into organoids |
| 1000 | 0 | 0 | 0 | 0 | 400 | cells don't cohere into organoids |
| 2000 | 0 | 0 | 0 | 0 | 0 | |
| 4400 | 0 | 0 | 0 | 0 | 0 | |
| 6000 | 0 | 0 | 0 | 0 | 0 | |

Withdrawing oxytocin also showed no effect, which is notable considering that oxytocin is regarded as necessary for mammary contractions in vivo (Richardson, 1949).

Contractile function implies the presence of contractility-associated structural proteins. Staining organoids with phalloidin showed cortical actin and, more specifically, cytokeratin 14+ cells stained positive for alpha-smooth muscle actin (ASMA) (FIG. 5G), a marker of the differentiated myoepithelium implicated in contractility. The association between contractility and ASMA expression was tested with inhibitor experiments. Contractile organoids were treated with either latrunculin B or jasplakinolide, inhibitors of cytoskeleton dynamics that either promote or inhibit actin polymerization, respectively. Both drugs were potent to inhibit contractions by >90% within 12 hours (FIG. 5H), suggesting the necessity of actomyosin for contractions. Next, organoids were treated with ML-7, an inhibitor of the smooth muscle myosin light-chain kinase that interacts with ASMA. Partial inhibition of contractions was observed with 2 μM ML-7 and >90% inhibition was observed with 50 μM ML-7 (FIG. 5I). This combination of experiments suggests that actomyosin in general and ASMA in particular are necessary for organoid contractions.

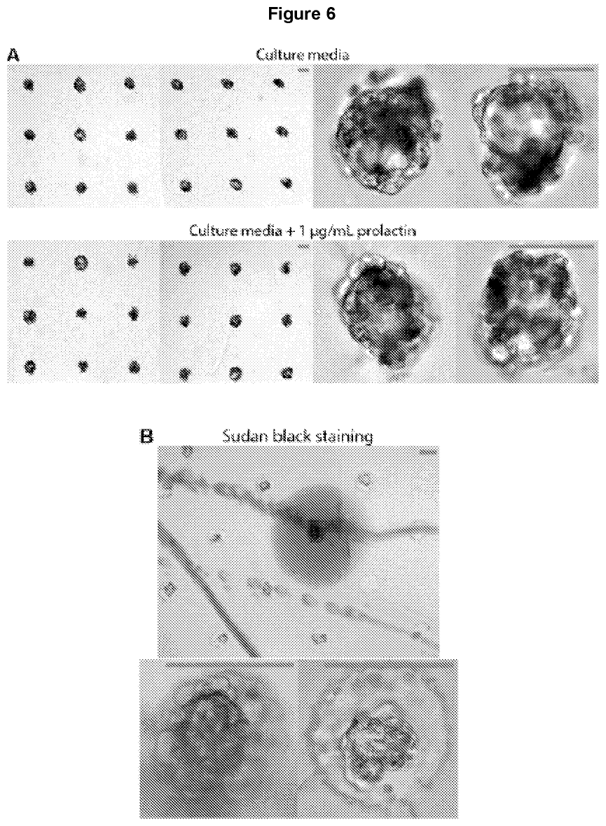

No luminal functional differentiation was detected beyond the expression of keratin 19 and MUC1 expression and the observation of lumens. Immunostaining failed to show expression of the estrogen receptor, and prolactin treatment failed to show significant evidence of lactation: no morphological changes were induced (FIG. 6A), with sudanophilia, a marker of lipophilic secretions (Dempsey et al., 1947), only evident in a single organoid (FIG. 6B). Using previously described methods (Fridriksdottir et al., 2015; Meng et al., 2019), induction of estrogen receptor expression was attempted. Fourth-passage HMECs were cultured in the presence of 25 μM RepSox and 10 μM SB431542 until confluence and then incorporated into microcontainer organoids. Four conditions were tested: maintaining the drugs during microcontainer culture, withdrawing the drugs upon microcontainer culture, mixing the cells 1:10 with undrugged cells, and an undrugged negative control. Estrogen receptor expression was not detected by immunofluorescence under any of these conditions. The evidence for increased functional differentiation was constrained to the MEP lineage.

### Example 4: Durability of Differentiated States

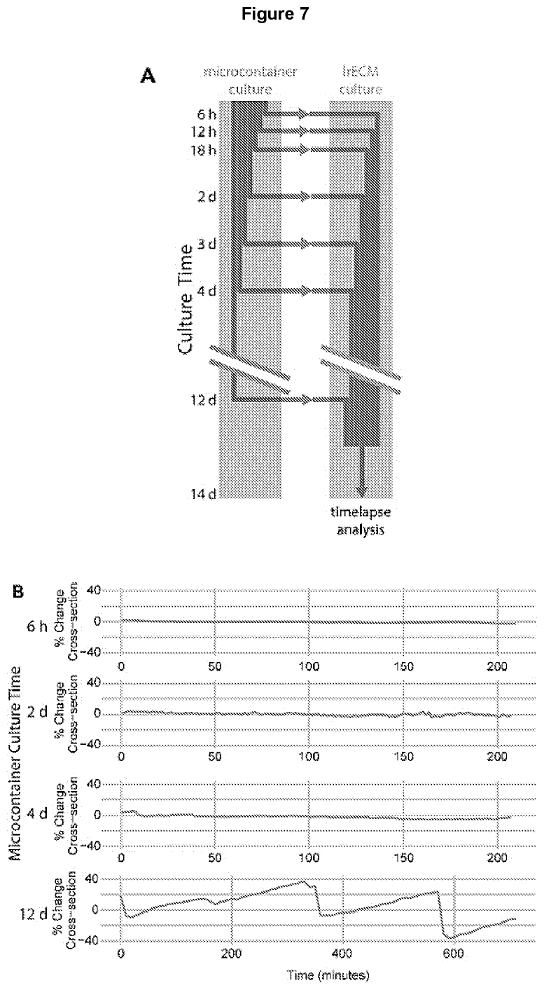

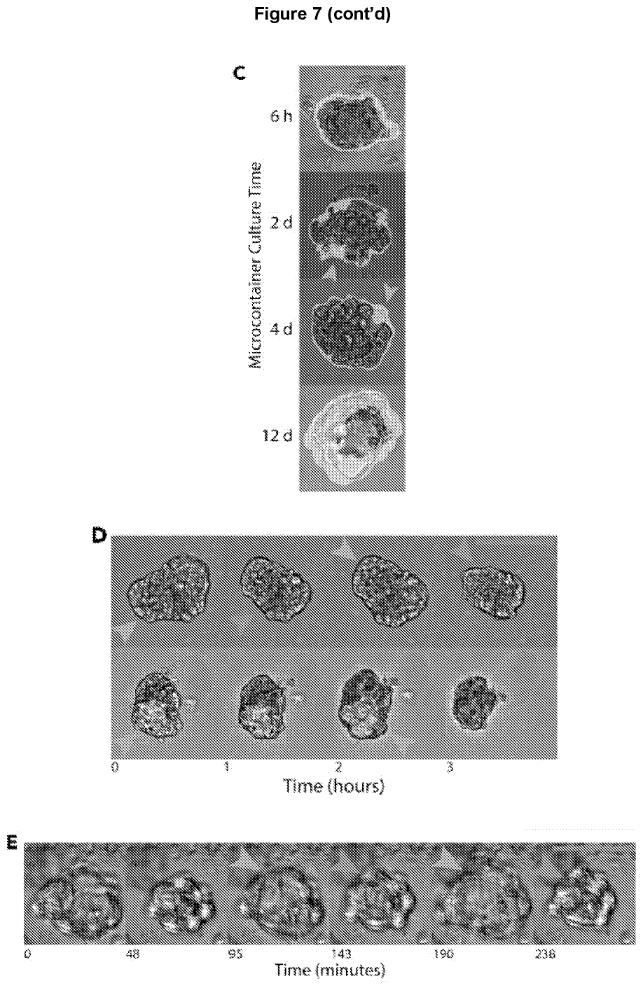

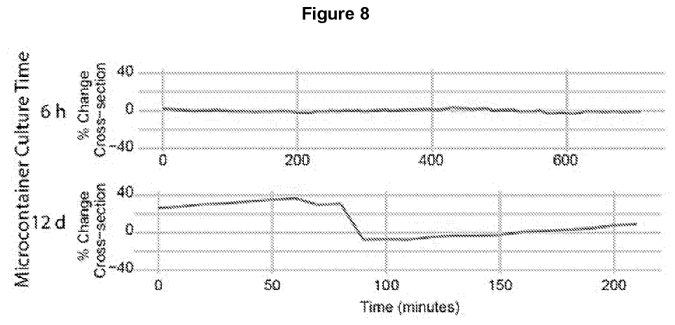

The stability of the contractile phenotype and its dependence on sustained microcontainer culture were investigated. To determine whether contractility could be sustained after organoids were removed from microcontainers, organoids were cultured in microcontainers and, after varied intervals, transferred out of the microcontainers and into embedded Matrigel culture (FIG. 7A).

After two weeks of culture, these organoids were imaged by time-lapse microscopy, and organoid contractility was assessed by cross-sectional area changes (FIG. 7B). Organoids kept in microcontainers for less than two days showed no contractility, organoids kept in microcontainers for up to four days showed contractility localized to small regions, and organoids kept in microcontainers for at least twelve days showed global contractility (FIG. 7C). In another experiment, organoids were cultured in microcontainers for two weeks and then transferred out of the microcontainers and into suspension culture, where they retained their contractility (FIG. 7D). Contractility could reliably be observed in long-term cultures, with contractions observed in many organoids kept in microcontainers for 53 days, as well as up to 157 days of sustained culture (FIG. 7E). Together, this evidence suggests that the initial period of microcontainer culture causes durable changes that are sufficient to establish metastable organoid behavior.

### Example 5: RNA-Seq Comparison of Microcontainer Organoids and Primary Cells

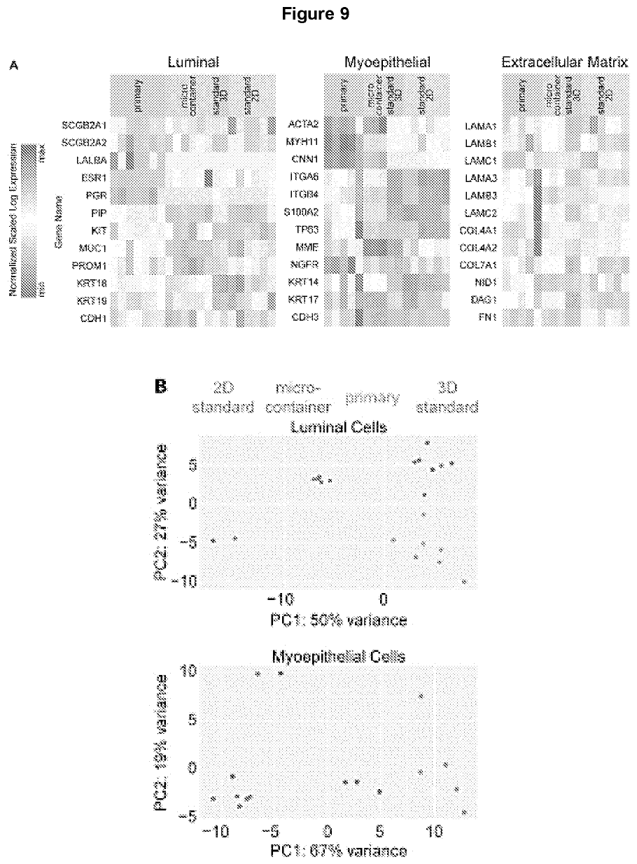

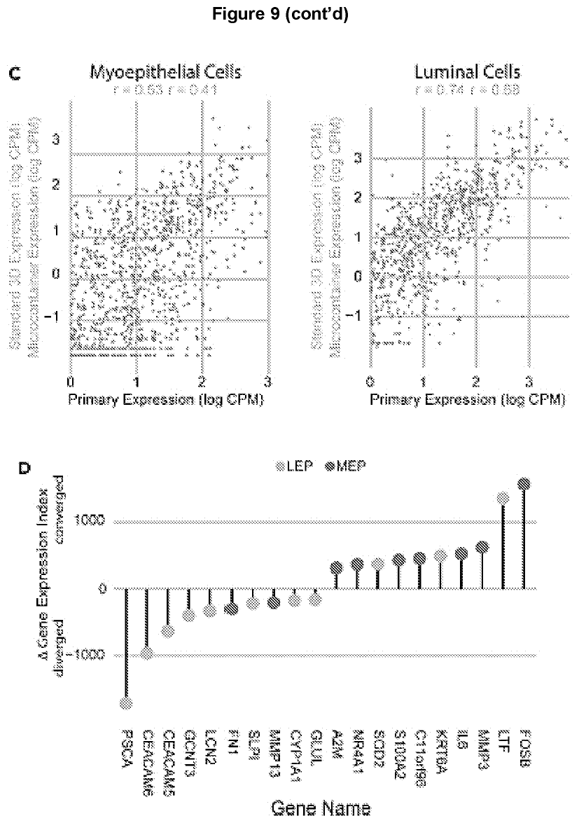

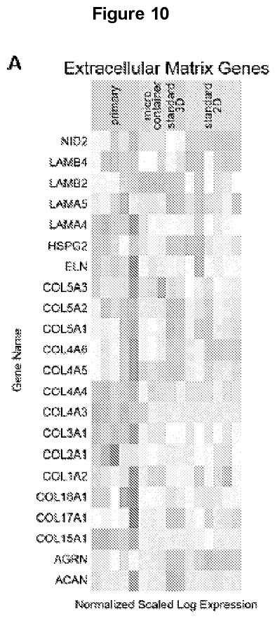

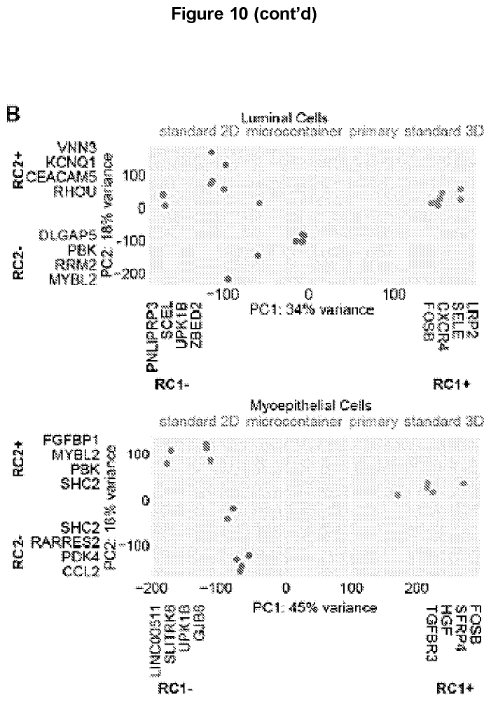

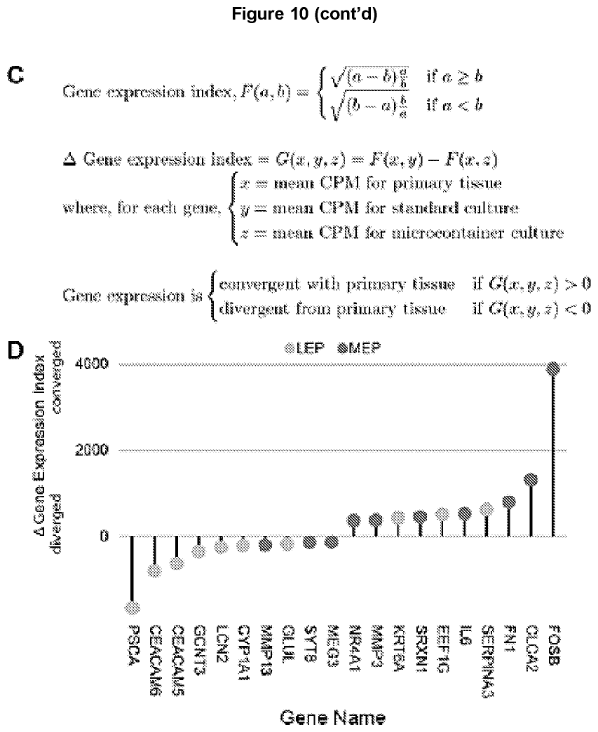

To better characterize the distinctiveness of microcontainer culture, RNA-seq was performed on MEP cells and LEP cells across several specimens from primary tissue, microcontainer culture, best practice three-dimensional culture (using on-top Matrigel format), and best practice two-dimensional cell culture. About 9600 organoids were harvested from microcontainers for the RNA isolation of each specimen. Expression was evaluated for defined markers of the LEP and MEP lineages (Sayaman et al., 2021), as well as for extracellular matrix proteins, especially basement membrane components (FIGS. 9A and 10A). MEP cells from microcontainers showed markedly increased expression of the contractility-associated genes ACTA2 andCNN1 (but not MYH11). MEP cells showed reduced expression of integrins ITGA6 and ITGB4 and lineage-specific keratins KRT18 and KRT19, bringing their expression closer to the low levels seen in primary tissue. LEP cells from microcontainers showed the same trend of reduced expression of lineage-specific keratin KRT14 (but not KRT17). These LEP cells also showed markedly increased expression of the progenitor-associated gene KIT and mucous barrier gene MUC1. The hormone receptors ESR1 and PGR, found in the primary tissue, were absent in LEP cells from standard cell culture and remained absent in microcontainers. Major components of the basement membrane—LAMA1, LAMB1, LAM C1, LAMA3, LAMB3, LAMC2, LAMA5, COL4A1, and COL4A2—were expressed under all culture conditions analyzed, suggesting that, although microcontainers may uniquely allow the accumulation of the associated gene products, the expression of these genes was not unique to any culture condition. Moreover, principal component analysis on these curated genes (FIG. 9B) showed clustering of microcontainer specimens proximal to primary specimens, especially for luminal cells.

The scope of the RNA-seq analysis was widened by calculating Pearson correlations on lineage-specific genes across primary tissue, microcontainer culture, and standard three-dimensional cell culture. To avoid spuriously inflating the Pearson correlation with housekeeping genes, lineage-specific genes were enriched using differential expression (DE) analysis between MEP cells and LEP cells via DESeq2 (Love et al., 2014), constraining correlation analysis to the 5157 genes with at least two-fold DE between MEP cells and LEP cells across the set of primary tissue (FIG. 9C). Although the resulting correlation coefficients appear to compare favorably with published Pearson-based analyses of mammary organoids with their tissues of origin (Rosenbluth et al., 2020), the absolute differences between Pearson correlation coefficients was small (r=0.53 for microcontainers vs r=0.41 for standard 3D for MEP cells and r=0.74 for microcontainers vs r=0.68 for standard 3D for LEP cells). However, this form of analysis may not have been sensitive enough to detect meaningful differences in gene expression patterns, so other techniques were used.

To further analyze differences across culture conditions, principal component and gene ontology analyses (Ashburner et al., 2000; The Gene Ontology Consortium, 2019) were performed on lineage-specific genes. Principal component analysis showed modest clustering of samples on the basis of culture conditions (FIG. 10B). To identify the genes with the greatest contributions to the principal components, varimax rotation was applied to the principal components, and the gene factors with the most extreme loadings for each rotated component were identified (FIG. 10B). The second varimax-rotated component (RC2) identified expressed genes shared by microcontainers and primary samples but not fourth-passage cells. Particularly notable among these genes, KCNQ1 stands out for its known role in apical ion transport into the mammary lumen (Abbott, 2014) and FDSCP stands out for its detectability in human milk (van Herwijnen et al., 2016) and casein homology (Kawasaki et al., 2011). As a complementary analysis to determine the most salient differences between standard 2D cell culture and microcontainer culture, genes were sorted by a gene expression distance index, calculated as the geometric mean of the additive and multiplicative gene expression differences versus primary tissue (FIG. 10C). With this index, positive values denote genes whose expression in microcontainers converges on primary tissue, and negative values denote genes whose expression in microcontainers diverges from primary tissue. The top ten most converged and diverged genes are shown in FIG. 9D. The same analysis was performed between standard 3D cell culture and microcontainer culture (FIG. 10D), but the smaller number of standard 3D specimens limited the interpretability of this analysis. To get a broader view of gene expression changes, the 200 most converged and hundred most diverged genes were analyzed by gene set analysis using PANTHER (Mi et al., 2019), with the statistically significant (false discovery rate-adjusted p<0.05) gene sets and fold enrichments shown in Table 6 and the breakdown of specific enrichments.

TABLE 6
Gene Set Enrichment Analysis

| | |
|---|---|
| Gene set | Fold enrichment |

Convergent gene expression

| | |
|---|---|
| Growth factor binding | 15 |
| Serine-type endopeptidase inhibitor^{a} | 10 |
| Protease binding^{a} | 7 |
| Carbohydrate derivative binding | 4 |

Divergent gene expression

| | |
|---|---|
| rRNA binding^{b} | 28 |
| Structural constituent of ribosome^{b} | 25 |
| Extracellular matrix binding | 24 |

RNA-seq analysis indicates that the expression of particular gene sets in microcontainer-based HME organoids converges or diverges from the expression in primary tissue specimens. The PANTHER Go-Slim Molecular Function gene sets were chosen for this analysis. Detail in Data S5.
^{a}The majority of matched genes in these sets overlap.
^{b}The majority of matched genes in these sets overlap.

This analysis shows that microcontainer organoids approach primary tissue with respect to serine protease inhibitors, as well as the binding of growth factors, proteases, and carbohydrate derivatives. In other respects, microcontainer organoids do not resemble primary tissue, with the predominant differences being in ribosomal and rRNA genes, as well as certain extracellular-matrix-binding genes.

### REFERENCES

The references, patents and published patent applications listed below, and all references cited in the specification above are hereby incorporated by reference in their entireties, as if fully set forth herein.
1. Abbott, G. W. (2014). Biology of the KCNQ1 potassium channel. New J. Sci. 2014.
2. Ashburner, M., Ball, C. A., Blake, J. A., Botstein, D., Butler, H., Michael Cherry, J., Davis, A. P., Dolinski, K., Dwight, S. S., Eppig, J. T., et al. (2000). Gene Ontology: tool for the unification of biology. Nat. Genet. 25, 25-29.
3. Boyer, P. M., and Hsu, J. T. (1992). Experimental studies of restricted protein diffusion in an agarose matrix. Aiche J. 38, 259-272.
4. Buchmann, B., Meixner, L. K., Fernandez, P., Hutterer, F. P., Raich, M. K., Scheel, C. H., and Bausch, A. R. (2019). Mechanical plasticity of the ECM directs invasive branching morphogenesis in human mammary gland organoids. bioRxiv. https://doi.org/10.1101/860015.
5. Cerchiari, A. E., Garbe, J. C., Jee, N. Y., Todhunter, M. E., Broaders, K. E., Peehl, D. M., Desai, T. A., LaBarge, M. A., Thomson, M., and Gartner, Z. J. (2015). A strategy for tissue self-organization that is robust to cellular heterogeneity and plasticity. Proc. Natl. Acad. Sci. USA 112, 2287-2292.
6. Cerchiari, A., Garbe, J. C., Todhunter, M. E., Jee, N. Y., Pinney, J. R., LaBarge, M. A., Desai, T. A., and Gartner, Z. J. (2014). Formation of spatially and geometrically controlled three-dimensional tissues in soft gels by sacrificial micromolding. Tissue Eng. Part C Methods 21, 541-547.
7. Chanson, L., Brownfield, D., Garbe, J. C., Kuhn, I., Stampfer, M. R., Bissell, M. J., and LaBarge, M. A. (2011). Self-organization is a dynamic and lineage-intrinsic property of mammary epithelial cells. Proc. Natl. Acad. Sci. USA 108, 3264-3269.
8. Chaudhuri, O., Koshy, S. T., Branco da Cunha, C., Shin, J.-W., Verbeke, C. S., Allison, K. H., and Mooney, D. J. (2014). Extracellular matrix stiffness and composition jointly regulate the induction of malignant phenotypes in mammary epithelium. Nat. Mater. 13, 970-978.
9. Corning. (n.d.). Corning Matrigel Matrix Frequently Asked Questions. https://www.corning.com/catalog/cls/documents/faqs/CLS-DL-CC-026.pdf.
10. Cruz-Acuña, R., and Garcia, A. J. (2017). Synthetic hydrogels mimicking basement membrane matrices to promote cell-matrix interactions. Matrix Biol. 57-58, 324-333.
11. Dempsey, E. W., Bunting, H., and Wislocki, G. B. (1947). Observations on the chemical cytology of the mammary gland. Am. J. Anat. 81, 309-341.
12. Djomehri, S. I., Burman, B., Gonzalez, M. E., Takayama, S., and Kleer, C. G. (2019). A reproducible scaffold-free 3D organoid model to study neoplastic progression in breast cancer. J. Cell Commun. Signal. 13, 129-143.
13. Duclut, C., Sarkar, N., Prost, J., and Jülicher, F. (2019). Fluid pumping and active flexoelectricity can promote lumen nucleation in cell assemblies. Proc. Natl. Acad. Sci. USA 116, 19264-19273.
14. Fridriksdottir, A. J., Kim, J., Villadsen, R., Klitgaard, M. C., Hopkinson, B. M., Petersen, O. W., and Rønnov-Jessen, L. (2015). Propagation of oestrogen receptorpositive and oestrogen-responsive normal human breast cells in culture. Nat. Commun. 6, 8786.
15. Fridriksdottir, A. J., Villadsen, R., Morsing, M., Klitgaard, M. C., Kim, J., Petersen, O. W., and Rønnov-Jessen, L. (2017). Proof of region-specific multipotent progenitors in human breast epithelia. Proc. Natl. Acad. Sci. USA 114, E10102-E10111.
16. Giannelli, G., Pozzi, A., Stetler-Stevenson, W. G., Gardner, H. A., and Quaranta, V. (1999). Expression of matrix metalloprotease-2-cleaved laminin-5 in breast remodeling stimulated by sex steroids. Am. J. Pathol. 154, 1193-1201.
17. Guzik, P., Piskorski, J., Krauze, T., Wykretowicz, A., and Wysocki, H. (2006). Heart rate asymmetry by Poincaré plots of RR intervals. Biomed. Tech. 51, 272-275.
18. Hansen, K. C., Kiemele, L., Maller, O., O'Brien, J., Shankar, A., Fornetti, J., and Schedin, P. (2009). An in-solution ultrasonication-assisted digestion method for improved extracellular matrix proteome coverage. Mol. Cell. Proteomics 8, 1648-1657.
19. Hassell, J. R., Robey, P. G., Barrach, H. J., Wilczek, J., Rennard, S. I., and Martin, G. R. (1980). Isolation of a heparan sulfate-containing proteoglycan from basement membrane. Proc. Natl. Acad. Sci. USA 77, 4494-4498.
20. Hidler, J. M., and Rymer, W. Z. (2000). Limit cycle behavior in spasticity: analysis and evaluation. IEEE Trans. Biomed. Eng. 47, 1565-1575.
21. Hindupur, S. K., Balaji, S. A., Saxena, M., Pandey, S., Sravan, G. S., Heda, N., Kumar, M. V., Mukherjee, G., Dey, D., and Rangarajan, A. (2014). Identification of a novel AMPK-PEA15 axis in the anoikis-resistant growth of mammary cells. Breast Cancer Res. 16, 420.
22. Kawasaki, K., Lafont, A.-G., and Sire, J.-Y. (2011). The evolution of milk casein genes from tooth genes before the origin of mammals. Mol. Biol. Evol. 28, 2053-2061.
23. Keller, F., Rudolf, R., and Hafner, M. (2019). Towards optimized breast cancer 3D spheroid mono- and co-culture models for pharmacological research and screening. J. Cell Biotechnol. 5, 89-101.
24. Kleinman, H. K., McGarvey, M. L., Hassell, J. R., Star, V. L., Cannon, F. B., Laurie, G. W., and Martin, G. R. (1986). Basement membrane complexes with biological activity. Biochemistry 25, 312-318.
25. Lategan, B. (n.d.). Normal histology of breast [WWW Document]. PathologyOutlines.com. http://www.pathologyoutlines.com/topic/breastnormal.html (accessed 4.20.20).
26. Love, M. I., Huber, W., and Anders, S. (2014). Moderated estimation of fold change and dispersion for RNA-seq data with DESeq2. Genome Biol. 15, 550.
27. Lutolf, M., Gjorevski, N. 2018. Three dimensional hydrogels for culturing organoids. US Patent. 20180258403:A1.
28. Meng, P., Vaapil, M., Tagmount, A., Loguinov, A., Vulpe, C., and Yaswen, P. (2019). Propagation of functional estrogen receptor positive normal human breast cells in 3D cultures. Breast Cancer Res. Treat. 176, 131-140.
29. Mi, H., Muruganujan, A., Ebert, D., Huang, X., and Thomas, P. D. (2019). PANTHER version 14: more genomes, a new PANTHER GO-slim and improvements in enrichment analysis tools. Nucleic Acids Res. 47, D419-D426.
30. Miyano, M., Sayaman, R. W., Stoiber, M. H., Lin, C.-H., Stampfer, M. R., Brown, J. B., and LaBarge, M. A. (2017). Age-related gene expression in luminal epithelial cells is driven by a microenvironment made from myoepithelial cells. Aging 9, 2026-2051.
31. Mroue, R., Inman, J., Mott, J., Budunova, I., and Bissell, M. J. (2015). Asymmetric expression of connexins between luminal epithelial- and myoepithelial-cells is essential for contractile function of the mammary gland. Dev. Biol. 399, 15-26.
32. Napolitano, A. P., Dean, D. M., Man, A. J., Youssef, J., Ho, D. N., Rago, A. P., Lech, M. P., and Morgan, J. R. (2007). Scaffold-free three-dimensional cell culture utilizing micromolded nonadhesive hydrogels. Biotechniques 43, 494, 496-500.
33. Nielsen, H. L., Gudjonsson, T., Villadsen, R., Rønnov-Jessen, L., and Petersen, O. W. (2003). Collagen gel contraction serves to rapidly distinguish epithelial- and mesenchymal-derived cells irrespective of a-smooth muscle actin expression. In Vitro Cell. Dev. Biol. Anim. 39, 297-303.
34. Okoh, V. O., Felty, Q., Parkash, J., Poppiti, R., and Roy, D. (2013). Reactive oxygen species via redox signaling to PI3K/AKT pathway contribute to the malignant growth of 4-hydroxy estradiol-transformed mammary epithelial cells. PLoS One 8, e54206.
35. Pelissier, F. A., Garbe, J. C., Ananthanarayanan, B., Miyano, M., Lin, C., Jokela, T., Kumar, S., Stampfer, M. R., Lorens, J. B., and LaBarge, M. A. (2014). Age-related dysfunction in mechanotransduction impairs differentiation of human mammary epithelial progenitors. Cell Rep. 7, 1926-1939.
36. Ricard-Blum, S. (2011). The collagen family. Cold Spring Harb. Perspect. Biol. 3, a004978.
37. Richardson, K. C. (1949). Contractile tissues in the mammary gland, with special reference to myoepithelium in the goat. Proc. R. Soc. Lond. B Biol. Sci. 136, 30-45.
38. Righetti, P. G., Brost, B. C. W., and Snyder, R. S. (1981). On the limiting pore size of hydrophilic gels for electrophoresis and isoelectric focussing. J. Biochem. Biophys. Methods 4, 347-363.
39. Rosenbluth, J. M., Schackmann, R. C. J., Gray, G. K., Selfors, L. M., Li, C. M.-C., Boedicker, M., Kuiken, H. J., Richardson, A., Brock, J., Garber, J., et al. (2020). Organoid cultures from normal and cancer-prone human breast tissues preserve complex epithelial lineages. Nat. Commun. 11, 1711.
40. Roskelley, C. D., and Bissell, M. J. (1995). Dynamic reciprocity revisited: a continuous, bidirectional flow of information between cells and the extracellular matrix regulates mammary epithelial cell function. Biochem. Cell Biol. 73, 391-397.
41. Ruiz-Herrero, T., Alessandri, K., Gurchenkov, B. V., Nassoy, P., and Mahadevan, L. (2017). Organ size control via hydraulically gated oscillations. Development 144, 4422-4427.
42. Sachs, N., de Ligt, J., Kopper, O., Gogola, E., Bounova, G., Weeber, F., Balgobind, A. V., Wind, K., Gracanin, A., Begthel, H., et al. (2018). A living biobank of breast cancer organoids captures disease heterogeneity. Cell 172, 373-386.e10.
43. Sayaman, R. W., Miyano, M., Senapati, P., Shalabi, S., Zirbes, A., Todhunter, M. E., Seewaldt, V., Neuhausen, S. L., Stampfer, M. R., Schones, D. E., and LaBarge, M. A. (2021). Epigenetic changes with age primes mammary luminal epithelia for cancer initiation. bioRxiv. https://doi.org/10. 1101/2021.02.12.430777.
44. Schedin, P., and Keely, P. J. (2011). Mammary gland ECM remodeling, stiffness, and mechanosignaling in normal development and tumor progression. Cold Spring Harb. Perspect. Biol. 3, a003228.
45. Stampfer, M. R., Vlodaysky, I., Smith, H. S., Ford, R., Becker, F. F., and Riggs, J. (1981). Fibronectin production by human mammary cells. J. Natl. Cancer Inst. 67, 253-261.
46. Stampfer, M. R., Yaswen, P., Alhadeff, M., and Hosoda, J. (1993). TGF beta induction of extracellular matrix associated proteins in normal and transformed human mammary epithelial cells in culture is independent of growth effects. J. Cell. Physiol. 155, 210-221.
47. Streuli, C. H., and Bissell, M. J. (1990). Expression of extracellular matrix components is regulated by substratum. J. Cell Biol. 110, 1405-1415.
48. Sumbal, J., Chiche, A., Charifou, E., Koledova, Z., and Li, H. (2020). Primary mammary organoid model of lactation and involution. Front. Cell Dev. Biol. 8, 68.
49. Taylor-Papadimitriou, J., Stampfer, M., Bartek, J., Lewis, A., Boshell, M., Lane, E. B., and Leigh, I. M. (1989). Keratin expression in human mammary epithelial cells cultured from normal and malignant tissue: relation to in vivo phenotypes and influence of medium. J. Cell Sci. 94 (Pt 3), 403-413.
50. The Gene Ontology Consortium (2019). The gene ontology resource: 20 years and still GOing strong. Nucleic Acids Res. 47, D330-D338.
51. Todhunter, M. E., Jee, N. Y., Hughes, A. J., Coyle, M. C., Cerchiari, A., Farlow, J., Garbe, J. C., LaBarge, M. A., Desai, T. A., and Gartner, Z. J. (2015). Programmed synthesis of three-dimensional tissues. Nat. Methods 12, 975-981.
52. van Herwijnen, M. J. C., Zonneveld, M. I., Goerdayal, S., Nolte-1 Hoen, E. N. M., Garssen, J., Stahl, B., Maarten Altelaar, A. F., Redegeld, F. A., and Wauben, M. H. M. (2016). Comprehensive proteomic analysis of human milk-derived extracellular vesicles unveils a novel functional proteome distinct from other milk components. Mol. Cell. Proteomics 15, 3412-3423.
53. Yu, L., Chen, M. C. W., and Cheung, K. C. (2010). Droplet-based microfluidic system for multicellular tumor spheroid formation and anticancer drug testing. Lab Chip 10, 2424-2432.
54. Yurchenco, P. D., Tsilibary, E. C., Charonis, A. S., and Furthmayr, H. (1985). Laminin polymerization in vitro. Evidence fora two-step assembly with domain specificity. J. Biol. Chem. 260, 7636-7644.
55. Zhao, L., Mok, S., and Moraes, C. (2019). Micropocket hydrogel devices for allin-one formation, assembly, and analysis of aggregate-based tissues. Biofabrication 11, 045013.
56. Garbe, J. C., Bhattacharya, S., Merchant, B., Bassett, E., Swisshelm, K., Feiler, H. S., Wyrobek, A. J., Stampfer, M. R., 2009. Molecular distinctions between stasis and telomere attrition senescence barriers shown by long-term culture of normal human mammary epithelial cells. Cancer Res. 69, 7557-7568.
57. Stampfer, M. R., Garbe, J. C., n.d. Human Mammary Epithelial Cell (HMEC) Bank, https://hmec.lbl.gov/

1. A method of producing an organoid without any exogenous extracellular matrix, comprising:
loading organ-specific cells in a microcontainer containing a culturing medium;
overlaying a hydrogel over the culture containing the cells such that the hydrogel forms a lid which is in direct contact with the surface of the culture to seal the culture; and
culturing the cells in the hydrogel-sealed microcontainer to obtain the organoid,
wherein the culturing medium does not contain any exogenous extracellular matrix.

2. A method of producing an organoid in the presence of a low concentration of exogenous extracellular matrix, comprising:
loading organ-specific cells in a microcontainer containing a culturing medium;
overlaying a hydrogel over the culture containing the cells such that the hydrogel forms a lid which is in direct contact with the surface of the culture to seal the culture; and
culturing the cells in the hydrogel-sealed microcontainer to obtain the organoid, wherein the culturing medium contains a concentration of exogenous extracellular matrix, lower than its minimum gelling concentration and insufficient to form a gel in the culturing medium.

3. The method of claim 2, wherein the culturing medium contains 0.5 mg/mL-1 mg/mL Matrigel.

4. The method of any one of claims 1-3, wherein the cells include epithelial cells and fibroblast cells.

5. The method of any one of claims 1-4, wherein the culturing medium has a higher density than the hydrogel lid.

6. The method of any one of claims 1-5, wherein the culturing medium has a density between about 1.1 g/ml and about 1.2 g/ml and the hydrogel lid has a density of about 1.0 g/ml.

7. The method of any one of claims 1-6, wherein the culturing medium comprises one or more biological colloids to achieve a higher density than the lid.

8. The method of claim 7, wherein the biological colloids include dextrin, maltodextrin, albumin, PEG-8000 and hydroxyethyl starch.

9. The method of claim 8, wherein the albumin includes bovine serum albumin or bovine serum albumin, fraction V.

10. The method of any one of claims 1-9, wherein the hydrogel comprises agarose.

11. An organoid produced by the method of any one of claims 1-10.

12. The organoid of claim 11, wherein the organoid exhibits contractility.

13. The organoid of claim 11 or claim 12, wherein the organoid exhibits pulsatile contractility.

14. A microcontainer for organoid culturing in the absence of any exogenous extracellular matrix, comprising walls composed of a hydrogel material and a hydrogel lid, wherein once cells and culturing medium are loaded in the microcontainer, the hydrogel walls and lid prevent the cells from escaping but allow air and liquid exchange with the environment.

15. The microcontainer of claim 14, wherein the hydrogel material includes agarose, gellan, alginate hydrogels or a combination thereof.

16. The microcontainer of claim 14 or claim 15, wherein the microcontainer has a diameter between about 100 μm and 150 μm.

17. The microcontainer of any one of claims 14-16, wherein the microcontainer has a depth between about 100 μm and 350 μm.

18. The microcontainer of any one of claims 14-17, wherein the microcontainer has a diameter of about 100 μm and a depth of about 200 μm.

19. The microcontainer of any one of claims 14-18, wherein the lid is in direct contact with the culturing medium containing cells once loaded with the culturing medium and the cells.
